SEQUENCE LISTING (31 sequences)

SEQ ID NO: 1 - protein, 22 aa, Artificial sequence
  synthetic peptide no. 1
KLYSPSQIGAFVLMKMKETAEN

SEQ ID NO: 2 - protein, 20 aa, Artificial sequence
  synthetic peptide no. 2
PSQIGAFVLMKMKETAENYL

SEQ ID NO: 3 - protein, 10 aa, Artificial sequence
  synthetic peptide no. 3
ATKDAGQISG

SEQ ID NO: 4 - protein, 18 aa, Artificial sequence
  synthetic peptide no. 4
PAYFNDSQRQATKDAGQI

SEQ ID NO: 5 - protein, 13 aa, Artificial sequence
  synthetic peptide no. 5
DSQRQATKDAGQI

SEQ ID NO: 6 - protein, 14 aa, Artificial sequence
  synthetic peptide no. 6
EPTAAALAYGLDKS

SEQ ID NO: 7 - protein, 20 aa, Artificial sequence
  synthetic peptide no. 7
GEDFDQALLRHIVKEFKRET

SEQ ID NO: 8 - protein, 18 aa, Artificial sequence
  synthetic peptide no. 8
NMALQRVREAAEKAKSEL

SEQ ID NO: 9 - protein, 23 aa, Artificial sequence
  synthetic peptide no. 9
PKHLNMKLTRAQFEGIVTDLIRR

SEQ ID NO: 10 - protein, 17 aa, Artificial sequence
  synthetic peptide no. 10
RAQFEGIVTDLIRRTIA

SEQ ID NO: 11 - protein, 8 aa, Artificial sequence
  synthetic peptide no. 11
PCQKAMQD

SEQ ID NO: 12 - protein, 14 aa, Artificial sequence
  synthetic peptide no. 12
MPKVQQTVQDLFGR

SEQ ID NO: 13 - protein, 17 aa, Artificial sequence
  synthetic peptide no. 13
PDEAVAIGAAIQGGVLA

SEQ ID NO: 14 - protein, 13 aa, Artificial sequence
  synthetic peptide no. 14
MVKNAEKYAEEDR

SEQ ID NO: 15 - protein, 30 aa, Artificial sequence
  synthetic peptide no. 15
RKKERVEAVNMAEGIIHDTETKMEEFKDQL

SEQ ID NO: 16 - protein, 25 aa, Artificial sequence
  synthetic peptide no. 16
FKDQLPADECNKLKEEISKMRELLA

SEQ ID NO: 17 - protein, 24 aa, Artificial sequence
  synthetic peptide no. 17
NKLKEEISKMRELLARKDSETGEN

SEQ ID NO: 18 - protein, 26 aa, Artificial sequence
  synthetic peptide no. 18
NIRQAASSLQQASLKLFEMAYKKMAS

SEQ ID NO: 19 - protein, 378 aa, Homo sapiens
  MISC_FEATURE: NBD domain 56-433 of RefSeq Accession No NP_004125 (SEQ ID NO 24)
VGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVT

SEQ ID NO: 20 - protein, 155 aa, Homo sapiens
  MISC_FEATURE: SBD domain 434-588 of RefSeq Accession No NP_004125 (SEQ ID NO 24)
DVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGI

SEQ ID NO: 21 - protein, 91 aa, Homo sapiens
  MISC_FEATURE: oligomerization domain 589-679 of RefSeq Accession No NP_004125 (SEQ ID NO 24)
IHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 22 - protein, 25 aa, Artificial sequence
  synthetic peptide  (TAT modified peptide 14)
MVKNAEKYAEEDRYMGRKKRRQRRR

SEQ ID NO: 23 - protein, 679 aa, homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 24 - protein, 679 aa, homo sapiens; identical to SEQ ID NO: 23

SEQ ID NO: 25 - DNA, 3506 bp, Homo sapiens
ttcctcccctggactctttctgagctcagagccgccgcagccgggacaggagggcaggctttctccaaccatcatgctgcggagcatattacctgtacgccctggctccgggagcggcagtcgagtatcctctggtcaggcggcgcgggcggcgcctcagcggaagagcgggcctctgggccgcagtgaccaacccccgcccctcaccccacgtggttggaggtttccagaagcgctgccgccaccgcatcgcgcagctctttgccgtcggagcgcttgtttgctgcctcgtactcctccatttatccgccatgataagtgccagccgagctgcagcagcccgtctcgtgggcgccgcagcctcccggggccctacggccgcccgccaccaggatagctggaatggccttagtcatgaggcttttagacttgtttcaaggcgggattatgcatcagaagcaatcaagggagcagttgttggtattgatttgggtactaccaactcctgcgtggcagttatggaaggtaaacaagcaaaggtgctggagaatgccgaaggtgccagaaccaccccttcagttgtggcctttacagcagatggtgagcgacttgttggaatgccggccaagcgacaggctgtcaccaacccaaacaatacattttatgctaccaagcgtctcattggccggcgatatgatgatcctgaagtacagaaagacattaaaaatgttccctttaaaattgtccgtgcctccaatggtgatgcctgggttgaggctcatgggaaattgtattctccgagtcagattggagcatttgtgttgatgaagatgaaagagactgcagaaaattacttggggcacacagcaaaaaatgctgtgatcacagtcccagcttatttcaatgactcgcagagacaggccactaaagatgctggccagatatctggactgaatgtgcttcgggtgattaatgagcccacagctgctgctcttgcctatggtctagacaaatcagaagacaaagtcattgctgtatatgatttaggtggtggaacttttgatatttctatcctggaaattcagaaaggagtatttgaggtgaaatccacaaatggggataccttcttaggtggggaagactttgaccaggccttgctacggcacattgtgaaggagttcaagagagagacaggggttgatttgactaaagacaacatggcacttcagagggtacgggaagctgctgaaaaggctaaatgtgaactctcctcatctgtgcagactgacatcaatttgccctatcttacaatggattcttctggacccaagcatttgaatatgaagttgacccgtgctcaatttgaagggattgtcactgatctaatcagaaggactatcgctccatgccaaaaagctatgcaagatgcagaagtcagcaagagtgacataggagaagtgattcttgtgggtggcatgactaggatgcccaaggttcagcagactgtacaggatctttttggcagagccccaagtaaagctgtcaatcctgatgaggctgtggccattggagctgccattcagggaggtgtgttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgtcagggtgaaagagagatggctggagacaacaaactccttggacagtttactttgattggaattccaccagcccctcgtggagttcctcagattgaagttacatttgacattgatgccaatgggatagtacatgtttctgctaaagataaaggcacaggacgtgagcagcagattgtaatccagtcttctggtggattaagcaaagatgatattgaaaatatggttaaaaatgcagagaaatatgctgaagaagaccggcgaaagaaggaacgagttgaagcagttaatatggctgaaggaatcattcacgacacagaaaccaagatggaagaattcaaggaccaattacctgctgatgagtgcaacaagctgaaagaagagatttccaaaatgagggagctcctggctagaaaagacagcgaaacaggagaaaatattagacaggcagcatcctctcttcagcaggcatcactgaagctgttcgaaatggcatacaaaaagatggcatctgagcgagaaggctctggaagttctggcactggggaacaaaaggaagatcaaaaggaggaaaaacagtaataatagcagaaattttgaagccagaaggacaacatatgaagcttaggagtgaagagacttcctgagcagaaatgggcgaacttcagtctttttactgtgtttttgcagtattctatatataatttccttaatttgtaaatttagtgaccattagctagtgatcatttaatggacagtgattctaacagtataaagttcacaatattctatgtccctagcctgtcatttttcagctgcatgtaaaaggaggtaggatgaattgatcattataaagatttaactattttatgctgaagtgaccatattttcaaggggtgaaaccatctcgcacacagcaatgaaggtagtcatccatagacttgaaatgagaccacatatggggatgagatccttctagttagcctagtactgctgtactggcctgtatgtacatggggtccttcaactgaggccttgcaagtcaagctggctgtgccatgtttgtagatggggcagaggaatctagaacaatgggaaacttagctatttatattaggtacagctattaaaacaaggtaggaatgaggctagacctttaacttccctaaggcatacttttctagctaccttctgccctgtgtctggcacctacatccttgatgattgttctcttacccattctggaatttttttttttttaaataaatacagaaagcatcttgatctcttgtttgtgaggggtgatgccctgagatttagcttcaagaatatgccatggctcatgcttcccatatttcccaaagagggaaatacaggatttgctaacactggttaaaaatgcaaattcaagatttggaagggctgttataatgaaataatgagcagtatcagcatgtgcaaatcttgtttgaaggattttattttctccccttagacctttggtacatttagaatcttgaaagtttctagatctctaacatgaaagtttctagatctctaacatgaaagtttttagatctctaacatgaaaaccaaggtggctattttcaggttgctttcagctccaagtagaaataaccagaattggcttacattaaagaaactgcatctagaaataagtcctaagatactatttctatggctcaaaaataaaaggaacccagatttctttcccta

SEQ ID NO: 26 - protein, 12 aa, Artificial sequence
  synthetic peptide (CELL PENETRATION SEQUENCE (derived from HIV TAT))
YMGRKKRRQRRR

SEQ ID NO: 27 - protein, 17 aa, Artificial sequence
  synthetic peptide
KAMQDAEVSKSDIGEVI

SEQ ID NO: 28 - protein, 17 aa, Artificial sequence
  synthetic peptide
QDLFGRAPSKAVNPDEA

SEQ ID NO: 29 - protein, 18 aa, Artificial sequence
  synthetic peptide no. 8′
NMALQRVREAAEKAKCEL

SEQ ID NO: 30 - protein, 13 aa, Artificial sequence
  synthetic peptide (scrambled - negative control)
KERYNEAKEDMVA

SEQ ID NO: 31 - protein, 559 aa, homo sapiens
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

(19) United States
(12) Patent Application Publication
(10) Pub. No.: US 2017/0355736 A1
(43) Pub. Date: Dec. 14, 2017

(54) MORTALIN PEPTIDES AND ANTIBODIES AND USES THEREOF FOR INHIBITING MORTALIN ACTIVITY AND TREATING A DISEASE ASSOCIATED WITH A PATHOLOGICAL CELL

(71) Applicant: Ramot at Tel-Aviv University Ltd., Tel-Aviv (IL)

(72) Inventor: Zvi FISHELSON, Tel-Aviv (IL)

(21) Appl. No.: 15/527,737

(22) PCT Filed: Nov. 19, 2015
(86) PCT No.: PCT/IL2015/051119

§ 371 Date: May 18, 2017

Related U.S. Application Data
(60) Provisional application No. 62/082,193, filed on Nov. 20, 2014.

(51) Int. Cl. C07K 14/47; A61K 38/17; C07K 16/18

(52) U.S. Cl. CPC C07K 14/4702; C07K 16/18; A61K 38/1709; C07K 2317/734

(57) ABSTRACT

Isolated mortalin peptides and antibodies are provided. Accordingly there is provided an isolated peptide comprising no more than 30 amino acids having a mortalin amino acid sequence and being capable of killing cancer cells and/or enhancing complement activity; also provided is an antibody comprising an antigen recognition domain having an amino acid sequence which binds mortalin and enhances complement-dependent cytotoxicity (CDC). Also provided are compositions and methods for inhibiting mortalin activity, killing a cell and treating a disease associated with a pathological cell population.

### FIELD AND BACKGROUND OF THE INVENTION

The present invention, in some embodiments thereof, relates to mortalin peptides and antibodies and, more particularly, but not exclusively, to the use of same for inhibiting mortalin activity and treating a disease associated with a pathological cell population.

Mortalin, also known as GRP75, PBP74, mitochondrial HSP75 and mot-2, is a member of the HSP70 family of proteins. Mortalin is ubiquitously and constitutively expressed in all eukaryotic cells. The Expression of mortalin is not heat-induced, yet may be affected by ionizing radiation, glucose deprivation and calorie restriction. It is mostly expressed in the mitochondria but also in the cytoplasm, endoplasmic reticulum, cytoplasmic vesicles and other compartments. Mortalin consists of three major functional domains: an N-terminal Nucleotide Binding Domain (NBD) which serves as an ATPase, a C-terminal Substrate Binding Domain (SBD) and a postulated oligomerization domain named ‘the lid” [Kaul and Wadhwa, Mortalin Biology: Life, Stress and Death (2012) XIV, 342 p., Chapter 2].

Mortalin has several binding partners and has been implicated in various functions ranging from cellular homeostasis, stress response, glucose regulation, intracellular and membrane trafficking, mitochondrial biogenesis, mitochondrial import and export, p53 inactivation, inhibition of complement activity, control of cell proliferation, differentiation, apoptosis, tumorigenesis, and viral release regulation.

Down-regulation of mortalin expression with antisense oligomers causes senescence-like growth arrest in immortalized cells, as reported by Wadhwa, R. et al., (2004) J Gene Med 6:439. Conversely, over expression of mortalin was documented to support cell survival and to promote tumorigenesis. Indeed, mortalin is over-expressed in several human tumors, such as neuroblastoma, lung adenocarcinoma, colorectal adenocarcinoma, lymphoma, leukemia and ovarian cancer [e.g., Kaul et al. Experimental Gerontology (2007) 42: 263-274]. Over expression of mortalin is also evident during infection and inflammation [e.g. Kirmanoglou, K. et al., Basic Res Cardiol (2004) 99:404 and Johannesen, J. et al., Autoimmunity (2004) 37:423].

Mortalin may support tumor cells survival in a variety of mechanisms. Thus, for example, mortalin plays a role in protection of cancer cells from complement-dependent cytotoxicity (CDC) and it facilitates removal of the complement membrane attack complex (MAC) from the cell surface by exo-vesiculation. Moreover, mortalin interacts with C8 and C9 causing a direct blocking of MAC incorporation into membranes. Indeed, mortalin inhibitors, such as siRNA and MKT-077, sensitize cells to CDC and inhibit the shedding of mortalin with the MAC. [Pilzer et al. Int Immunol (2005) 17:1239-1248; Pilzer et al., Springer Semin Immunopathol (2005) 27:375-387; Pilzer et al. Int J Cancer (2010). 126:1428-1435 and Saar Ray et al. J Biol Chem. (2014) 289(20:15014-22].

In addition, mortalin and cytoplasmic p53 co-localize and interact together resulting in higher mortalin-p53 association levels in cancer cell lines and tumor models. This interaction promotes sequestration of p53 in the cytoplasm, and inhibits normal transcriptional activation function of p53 [Kaul et al. Experimental Gerontology (2007) 42: 263-274].

Several approaches have been proposed for decreasing the levels/activity of mortalin for treating various medical conditions associated with the detrimental activity of mortalin including, inhibitory small molecules such as MKT-077 and SHetA2; deoxyspergualin; ribozymes; anti-sense RNA and anti-mortalin antibodies [e.g. International Patent Application Publication No: WO2008032324; U.S. Pat. No. 8,293,716 and U.S. Pat. No. 8,470,793; Wadhwa et al., Cancer Research (2000) 60; 6818-6821; Benbrook et al. Invest New Drugs. (2014) 32(3): 412-423; Didik et al. Bioinformation. (2012) 8(9): 426-429; Wadhwa et al., J Gene Med. (2004) 6: 439-44; Fishelson Z. et al., Int Immunol. (2001) 13: 983-991 and Wadhwa et al., (2003) EMBO Rep 4: 59 5-601].

Additional background art include, U.S. Pat. No. 8,586,042; U.S. Pat. No. 5,627,039; U.S. Pat. No. 7,700,307; U.S. Pat. No. 8,293,716; U.S. Patent Application publication No. 20060270622; and U.S. Patent Application publication No. 20120302729.

### SUMMARY OF THE INVENTION

According to an aspect of some embodiments of the present invention there is provided an isolated peptide comprising no more than 100 amino acids having a mortalin amino acid sequence and being capable of killing cancer cells.

According to an aspect of some embodiments of the present invention there is provided an isolated peptide comprising no more than 100 amino acids having a mortalin amino acid sequence and being capable of enhancing complement activity.

According to an aspect of some embodiments of the present invention there is provided an antibody comprising an antigen recognition domain having an amino acid sequence which binds a mortalin peptide and enhances complement-dependent cytotoxicity (CDC), wherein said mortalin peptide is selected from the group consisting of SEQ ID NOs: 1-10, 13-18 and 29.

According to an aspect of some embodiments of the present invention there is provided a method of inhibiting mortalin activity, the method comprising contacting cells which express mortalin with the isolated peptide of some embodiments of the invention or with the antibody of some embodiments of the invention, thereby inhibiting mortalin activity.

According to an aspect of some embodiments of the present invention there is provided a method of killing a cell, the method comprising contacting a cell which expresses mortalin with the isolated peptide of some embodiments of the invention or with the antibody of some embodiments of the invention, thereby killing the cell.

According to an aspect of some embodiments of the present invention there is provided a method of treating a disease associated with a pathological cell population in a subject in need thereof, the method comprising administering to the subject a therapeutically effective amount of the isolated peptide of some embodiments of the invention or the antibody of some embodiments of the invention, thereby treating the disease associated with a pathological cell population.

According to an aspect of some embodiments of the present invention there is provided a use of the isolated peptide of some embodiments of the invention or the antibody of some embodiments of the invention for the manufacture of a medicament identified for the treatment of a disease associated with a pathological cell population.

According to an aspect of some embodiments of the present invention there is provided a pharmaceutical composition comprising as an active ingredient the isolated peptide of some embodiments of the invention or the antibody of some embodiments of the invention, and a pharmaceutically acceptable carrier or diluent.

According to an aspect of some embodiments of the present invention there is provided an article of manufacture identified for treatment of a disease associated with a pathological cell population comprising packaging material packaging the isolated peptide of some embodiments of the invention or the antibody of some embodiments of the invention, and an antibody capable of specifically binding the pathological cell population.

According to some embodiments of the invention, the peptide comprises a mortalin amino acid sequence with the proviso that the peptide does not comprise the amino acid sequence depicted in SEQ ID NO: 27 (KAMQDAEVSKSDIGEVI) or SEQ ID NO: 28 (QDLFGRAPSKAVNPDEA).

According to some embodiments of the invention, the killing of said cancer cells is complement-dependent.

According to some embodiments of the invention, the amino acid sequence is capable of enhancing complement activity.

According to some embodiments of the invention, the killing of said cancer cells is complement-independent.

According to some embodiments of the invention, the enhancing complement activity is via inhibiting binding of mortalin to C9.

According to some embodiments of the invention, the enhancing complement activity is via reducing mortalin-induced inhibition of C9 polymerization.

According to some embodiments of the invention, the enhancing complement activity is via enhancing complement-dependent cytotoxicity (CDC).

According to some embodiments of the invention, the peptide is capable of enhancing complement-independent cytotoxicity.

According to some embodiments of the invention, the mortalin amino acid sequence comprises at least a portion of a nucleotide binding domain (NBD) of mortalin with the proviso that the peptide does not comprise the amino acid sequence depicted in SEQ ID NO: 27 (KAMQDAEVSKSDIGEVI) or SEQ ID NO: 28 (QDLFGRAPSKAVNPDEA).

According to some embodiments of the invention, the peptide comprises the amino acid sequence set forth by SEQ ID NO:2.

According to some embodiments of the invention, the peptide comprises an amino acid sequence selected from the group consisting of SEQ ID NOs: 1-13 and 29.

According to some embodiments of the invention, the mortalin amino acid sequence comprises at least a portion of a substrate binding domain (SBD) of mortalin.

According to some embodiments of the invention, the peptide comprises an amino acid sequence selected from the group consisting of SEQ ID NO: 14-15.

According to some embodiments of the invention, the mortalin amino acid sequence comprises at least a portion of an oligomerization domain of mortalin.

According to some embodiments of the invention, the peptide comprises an amino acid sequence selected from the group consisting of SEQ ID NO: 15-18.

According to some embodiments of the invention, the peptide comprises an amino acid sequence selected from the group consisting of SEQ ID NOs: 1-18 and 29.

According to some embodiments of the invention, the peptide comprises an amino acid sequence selected from the group consisting of SEQ ID NOs: 2, 7, 8, 10, 14, and 16.

According to some embodiments of the invention, the peptide comprises an amino acid sequence selected from the group consisting of SEQ ID NO: 1, 2, 7, 9, 10, 14, 15 and 16.

According to some embodiments of the invention, the peptide comprises an amino acid sequence selected from the group consisting of SEQ ID NO: 2, 9, 10, 14, 15, and 16.

According to some embodiments of the invention, the peptide comprises no more than 30 amino acids.

According to some embodiments of the invention, the peptide is attached to a cell penetrating agent.

According to some embodiments of the invention, the cell penetrating agent comprises an amino acid sequence derived from the HIV TAT polypeptide.

According to some embodiments of the invention, the amino acid sequence derived from the HIV TAT polypeptide is depicted in SEQ ID NO: 26.

According to some embodiments of the invention, the isolated peptide is set forth by SEQ ID NO:2.

According to some embodiments of the invention, the antibody is a monoclonal antibody.

According to some embodiments of the invention, the mortalin activity is complement dependent.

According to some embodiments of the invention, the mortalin activity is complement independent.

According to some embodiments of the invention, the killing is complement dependent.

According to some embodiments of the invention, the killing is complement independent.

According to some embodiments of the invention, the ability of the isolated peptide to kill the cell is determined by presence of mitochondrial damage.

According to some embodiments of the invention, the mitochondrial damage comprises depolarization of the mitochondrial membrane.

According to some embodiments of the invention, detecting depolarization of the mitochondrial membrane is performed by monitoring the decrease in the red/green fluorescence intensity ratio emitted by the JC-1 dye. According to some embodiments of the invention, the contacting is effected in-vivo.

According to some embodiments of the invention, the contacting is effected in-vitro or ex-vivo.

According to some embodiments of the invention, the cell is a pathological cell.

According to some embodiments of the invention, the method further comprising administering to the subject an antibody capable of specifically binding said pathological cell population.

According to some embodiments of the invention, the use further comprises an antibody capable of specifically binding said pathological cell population.

According to some embodiments of the invention, the pharmaceutical composition further comprises an antibody capable of specifically binding a pathological cell.

According to some embodiments of the invention, the peptide or antibody and said antibody capable of specifically binding the pathological cell population are packaged in separate containers.

According to some embodiments of the invention, the disease associated with a pathological cell population is selected from the group consisting of cancer, an infectious disease, an autoimmune disease and a transplantation-related disease.

According to some embodiments of the invention, the disease associated with the pathological cell population is cancer.

According to some embodiments of the invention, the pathological cell is cancer cell.

According to some embodiments of the invention, the cancer is lymphoma or leukemia.

According to some embodiments of the invention, the antibody capable of specifically binding the pathological cell population comprises an antibody constant region capable of initiating a CDC.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a histogram depicting results of ELISA (enzyme-linked immunosorbent assay) which detected binding of mortalin to C9 in the presence or absence of various mortalin peptides. Purified human C9 (0.057 μM) was attached overnight at 4° C. to wells of a 96-wells plate and then the excess of C9 was washed. Following, His-tagged recombinant mortalin (0.007 μM) was added to the wells for 1 hour at 37° C. with or without the designated peptides (each at a concentration of 170 μM), which were dissolved in 1% BSA (bovine serum albumin). Following incubation with the mortalin and peptides the wells were washed, treated for 1 hour at 37° C. with mouse anti-His antibody (diluted 1:1000) followed by 1 hour incubation at room temperature with peroxidase-labeled goat anti-mouse IgG (diluted 1:10,000), developed with O-phenylenediamine (OPD) and analyzed by an ELISA reader at 492 nm. The effect of mortalin peptides on C9-mortalin binding is shown by the increase or decrease as compared to the binding of the positive control in the absence of the mortalin peptide (first bar on the left).

FIGS. 2A-B are Coomassie blue stained gels depicting inhibition of C9 polymerization by mortalin or the isolated mortalin peptides. Purified human C9 (1.4 mM) was mixed for 15 minutes at 37° C. with BSA (1.4 mM) as control (lanes marked as “BSA”), mortalin (1.4 mM, lanes marked as “mot”) or mortalin peptides (1 mM, lanes marked by “p” and the indicated peptide numbers) that were freshly dissolved in Tris buffered saline (TBS); and then incubated with 42 μM ZnCl₂ for 2 hours at 37° C. The first lane on the left, marked “C9” refers to a positive control of the purified C9 without BSA, without mortalin or without the isolated peptides. Samples were subjected to SDS-PAGE on a 2.5-14% acrylamide gradient gel and stained with Coomassie blue. The bands of poly C9 appearing on the top part of the gel are indicated. FIGS. 2A and 2B represent two independent experiments.

FIGS. 3A-D depicts a histogram (FIG. 3A) and images (FIGS. 3B-D) demonstrating that the mortalin peptides are toxic to cancer cells. Raji or K562 cells were treated without (C) or with 2 mM peptides for 48 hours in their culture medium. Then, the cells were labeled with Annexin/PI and analyzed by Flow Cytometry. FIG. 3A—a histogram depicting the percent of dead cells (Annexin and PI positive) as calculated. FIGS. 3B-D—representative images of cells in control (FIG. 3B) and peptide-treated groups [FIG. 3C (treated with peptide no. 14) and FIG. 3D (treated with peptide no. 16)] stained by DAPI and observed under a fluorescence microscope.

FIG. 4 is a histogram depicting the percentage of cell death in cells treated with the mortalin peptides. Raji or K562 cells were treated with 0.8 mM of peptide no: 14 (SEQ ID NO: 14) or TAT-peptide no: 14 (SEQ ID NO: 22, a conjugated sequence of peptide no. 14 and the TAT-derived cell penetration sequence) for 24 hours in their culture medium. Following, the cells were labeled with Annexin/PI and analyzed by Flow Cytometry. Percent of dead cells (Annexin and PI positive) was calculated. Note that the addition of the cell penetration sequence (which increases permeability into the cells, e.g., the TAT sequence) results in increased toxicity of peptide no: 14 to cancer cells (e.g., at least 3 or 9 folds increase in the percentage of cell death).

FIG. 5 is a graph depicting titration of peptide toxic concentration of peptides no: 2, 7, 8, 10, 14 and 19 on Raji cancer cells. Shown is the percentage of Raji PI positive cells (dead cells) following treatment with increasing concentrations of the mortalin peptides. A peptide with a scrambled sequence was used as negative control. Raji cells were treated for 24 hours with the various peptides at the indicated concentrations in their culture medium. Following, the cells were labeled with PI and analyzed by Flow Cytometry. Percent of dead cells (PI positive) was calculated.

FIG. 6 is a graph depicting titration of peptide toxic concentration of peptides no: 2, 7, 8, 10, 14 and 19 on Ramos cancer cells. Shown is the percentage of Ramos PI positive cells (dead cells) following treatment with increasing concentrations of the mortalin peptides. A peptide with a scrambled sequence was used as negative control. Ramos cells were treated for 24 hours with the various peptides at the indicated concentrations in their culture medium. Following, the cells were labeled with PI and analyzed by Flow Cytometry. Percent of dead cells (PI positive) was calculated.

FIG. 7 is a graph depicting titration of peptide toxic concentration of peptides no: 2, 7, 8, 10, 14 and 19 on Z-138 cancer cells. Shown is the percentage of Z-138 PI positive cells (dead cells) following treatment with increasing concentrations of the mortalin peptides. A peptide with a scrambled sequence was used as negative control. Cells were treated for 24 hours with the various peptides at the indicated concentrations in their culture medium. Following, the cells were labeled with PI and analyzed by Flow Cytometry. Percent of dead cells (PI positive) was calculated.

FIG. 8 is a histogram depicting cell death of B-CLL leukemia cells by mortalin peptides. Shown is the percentage of PI positive cells (dead cells) following treatment with the indicated mortalin peptides at a concentration of 50 μM. Mortalin peptides numbers 2, 7, 8, 10, 14 and 16 and a scrambled control peptide (PC) were tested. B-CLL leukemia cells from patient number 1 (#1) were treated for 24 hours with the various peptides at 50 μM concentration in their culture medium. Following, the cells were labeled with PI and analyzed by Flow Cytometry. Percent of dead cells (PI positive) was calculated.

FIG. 9 is a histogram depicting cell death of B-CLL leukemia cells by mortalin peptides. Shown is the percentage of PI positive cells (dead cells) following treatment with the indicated mortalin peptides at a concentration of 50 μM. Mortalin peptides numbers 2, 7, 8, 10, 14 and 16 and a scrambled control peptide (PC) were tested. B-CLL leukemia cells from patient number 2 (#2) were treated for 24 hours with the various peptides at 50 μM concentration in their culture medium. Following, the cells were labeled with PI and analyzed by Flow Cytometry. Percent of dead cells (PI positive) was calculated.

FIG. 10 is a histogram depicting cell death of B-CLL leukemia cells by mortalin peptides. Shown is the percentage of PI positive cells (dead cells) following treatment with the indicated mortalin peptides at a concentration of 50 μM. Mortalin peptides numbers 2, 7, 8, 10, 14 and 16 and a scrambled control peptide (PC) were tested. B-CLL leukemia cells from patient number 3 (#3) were treated for 24 hours with the various peptides at 50 μM concentration in their culture medium. Following, the cells were labeled with PI and analyzed by Flow Cytometry. Percent of dead cells (PI positive) was calculated.

FIG. 11 is a histogram depicting cell death of B-CLL leukemia cells and normal mononuclear cells by mortalin peptides. Shown is the percentage of PI positive cells (dead cells) following treatment with the indicated mortalin peptides at a concentration of 50 μM. Mortalin peptides numbers 2, 7, 8, 10, 14 and 16 and a scrambled control peptide (PC) were tested. B-CLL leukemia cells from patient number 4 (#4) (blue bars) and normal mononuclear cells (healthy subject; red bars) were treated for 24 hours with the various peptides at 50 μM concentration in their culture medium. Following, the cells were labeled with PI and analyzed by Flow Cytometry. Percent of dead cells (PI positive) was calculated.

FIG. 12 is a histogram depicting the effect of the peptide of some embodiments of the invention on mitochondrial damage. Ramos cells were incubated for 1 hour at 37° C. with either 50 μM of scrambled peptide (PC; SEQ ID NO: 30) or with 50 μM peptide number 2 (P2, SEQ ID NO: 2) or 7 (P7, SEQ ID NO:7), or were left untreated. Following incubation, the cells were washed and a staining solution was added for 20 minutes at 37° C. Cells were washed and suspended in JC-1 staining buffer (Sigma) and analyzed by FACS. Red mean fluorescence intensity (MFI) was quantified. Shown are the red mean fluorescent intensity (MFI) in the treated or untreated cells. Note that upon treating the cells for 1 hour at 37° C. with P2 or P7 the mitochondrial membrane potential dissipated (red MFI decreased) relative to that of cells treated with a scrambled peptide (PC).

### DESCRIPTION OF SPECIFIC EMBODIMENTS OF THE INVENTION

The present invention, in some embodiments thereof, relates to mortalin peptides and antibodies and, more particularly, but not exclusively, to the use of same for inhibiting mortalin activity and treating a disease associated with a pathological cell population.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Mortalin, a member of the HSP70 family of proteins, has been implicated in various functions such as cellular homeostasis, stress response, mitochondrial biogenesis, mitochondrial import and export, p53 inactivation, inhibition of complement activity, control of cell proliferation, apoptosis and tumorigenesis. Over-expression of mortalin is evident in several human tumors and also during infection and inflammation. Several approaches have been proposed for decreasing the levels/activity of mortalin for treating various medical conditions associated with the detrimental activity of mortalin including, inhibitory small molecules such as MKT-077 and SHetA2; deoxyspergualin; ribozymes; anti-sense RNA and anti-mortalin antibodies.

The present inventor has generated novel mortalin-derived peptides and uncovered that these peptides can suppress mortalin activity and/or induce cell death in a complement-dependent and/or a complement-independent manner(s) and suggest their use in inhibiting mortalin activity, enhancing complement activity, killing a cell and/or treating a disease associated with pathological cells, such as cancer.

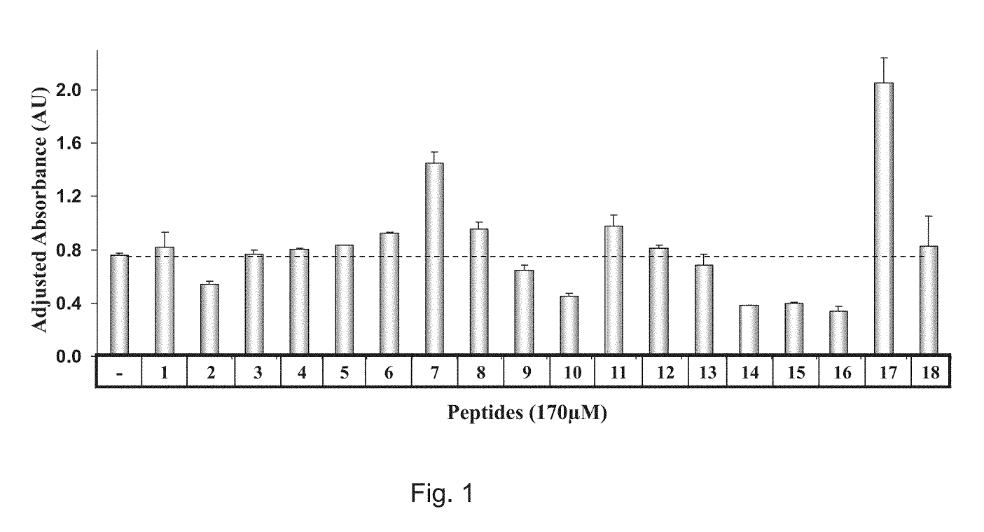

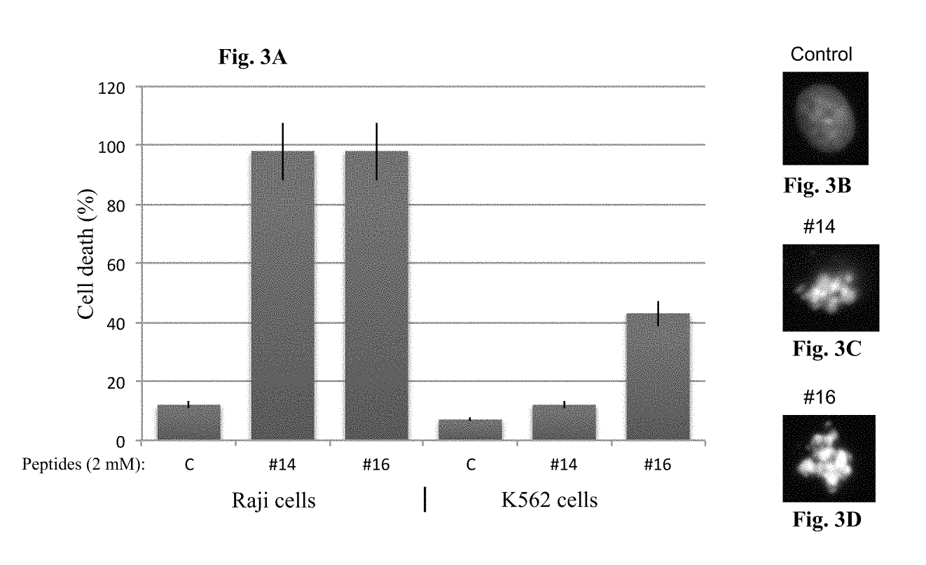

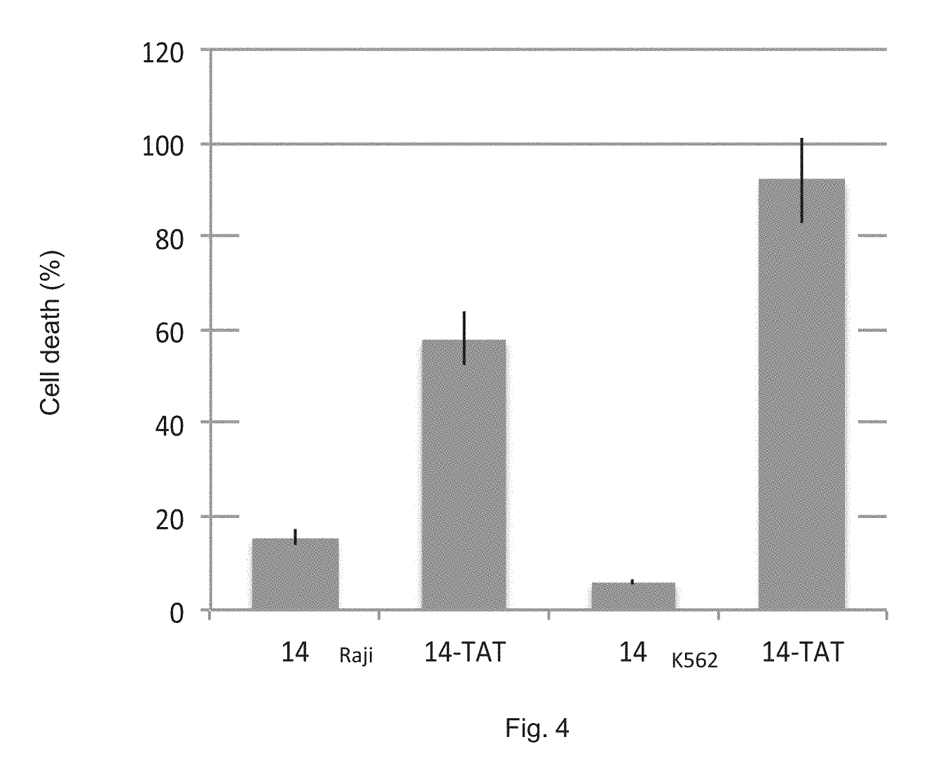

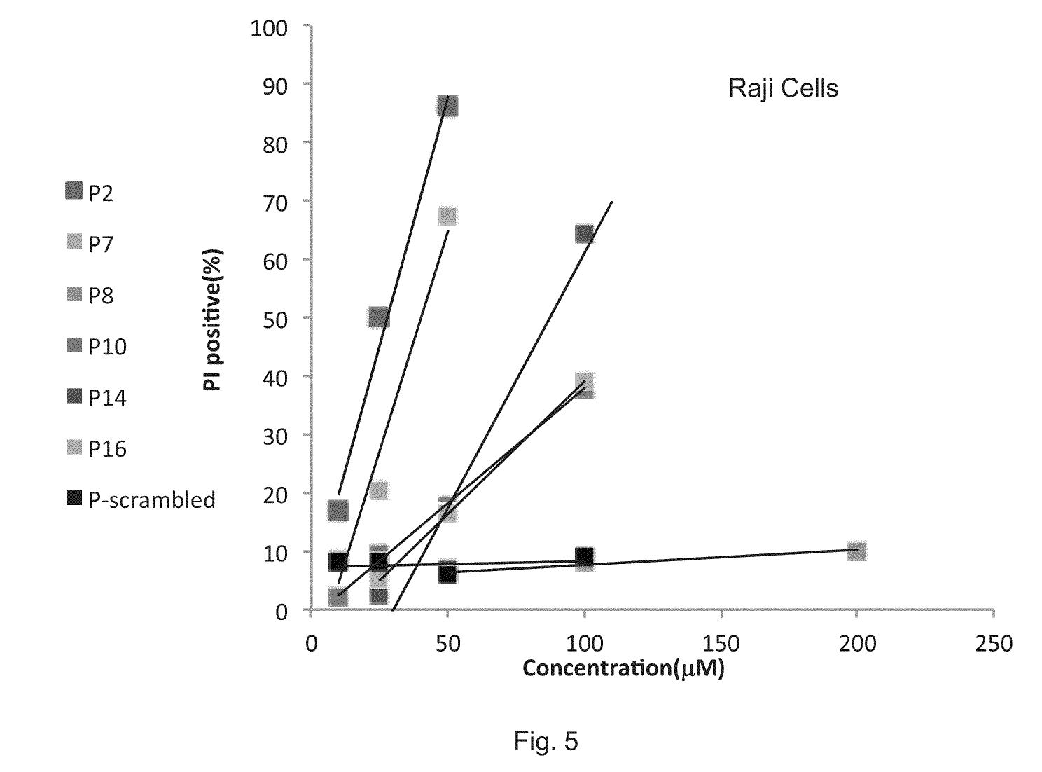

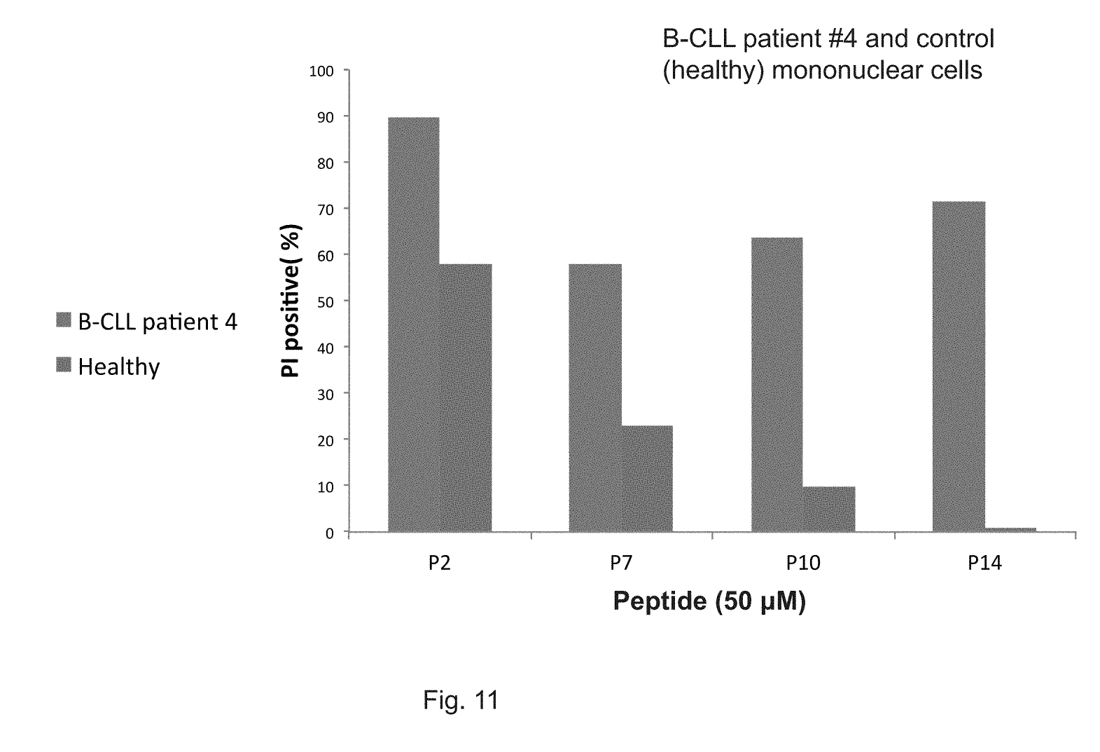

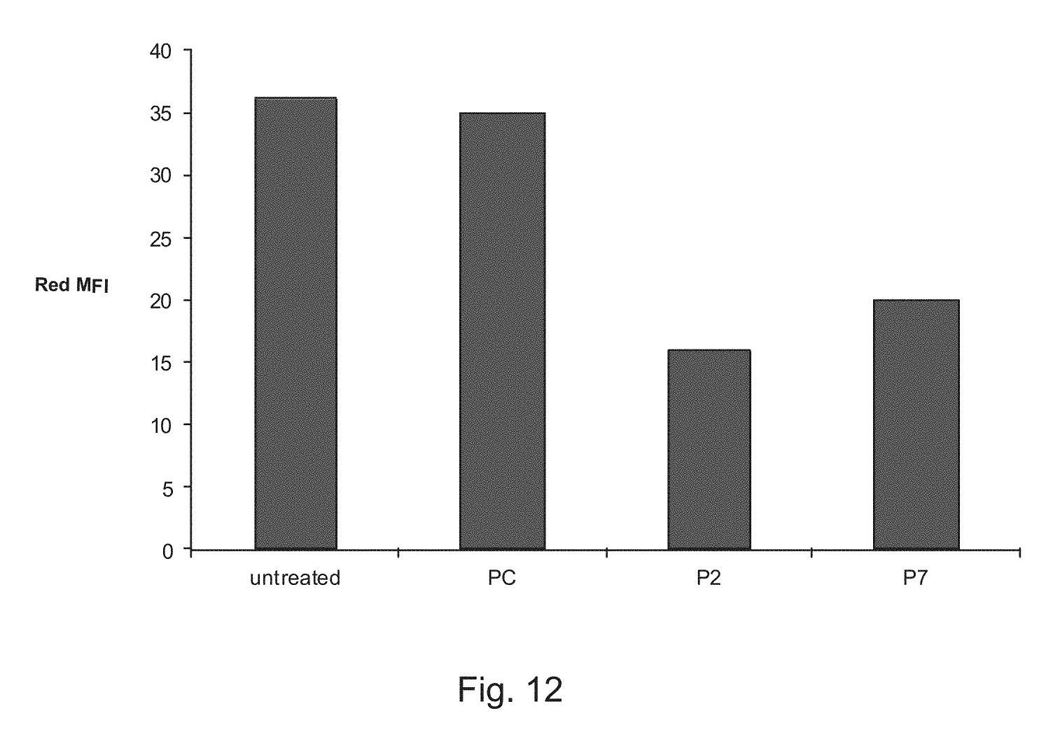

The Examples section which follows demonstrates that peptides derived from the mortalin protein (e.g., SEQ ID NO:24) can inhibit the binding of mortalin to the C9 protein (e.g., SEQ ID NO:31) of the complement (e.g., the peptides set forth by SEQ ID NOs: 2, 9, 10, 14, 15 and 16; FIG. 1, Example 1); can enhance complement-dependent cytotoxicity (CDC) (e.g., the peptides set forth by SEQ ID NOs: 1, 2, 7, 9, 10, 14, 15 and 16; Table 4, Example 3); as well as enhancing complement independent cytotoxicity (direct cell killing; e.g., the peptides set forth by SEQ ID NOs: 2, 7, 8, 10, 14 and 16; FIGS. 5-11, Table 6, Example 5). In addition, as shown in Examples 4 and 5 of the Examples section which follows while some of the peptides exhibit high (e.g., the peptides set forth by SEQ ID NOs: 2, 7, 15 and 16), moderate (e.g., the peptides set forth by SEQ ID NOs: 1 and 17) or low (e.g., the peptides set forth by SEQ ID NOs: 9, 10 and 14) ability to penetrate into cells, the ability to penetrate the cells can significantly increase by attaching a cell penetration agent (e.g., the TAT derived sequence set forth by SEQ ID NO: 26) to thereby increase the entrance into the cell(s) and accordingly the cell killing ability of the peptides of some embodiments of the invention (FIGS. 3 and 4). Moreover, as shown in Example 6 of the Examples section which follows, the peptides of some embodiments of the invention caused mitochondrial damage to the cancer cells (FIG. 12).

According to an aspect of the present invention there is provided an isolated peptide comprising no more than 100 amino acids having a mortalin amino acid sequence and being capable of killing cancer cells.

According to an aspect of the present invention there is provided an isolated peptide comprising no more than 100 amino acids having a mortalin amino acid sequence and being capable of enhancing complement activity.

As used herein the term “isolated” refers to at least partially separated from the natural environment e.g., from a human cell.

As used herein “mortalin” also known as HSPA9; CSA; GRP-75; GRP75; HEL-S-124m; HSPA9B; MOT; MOT2; MTHSP75; PBP74; and mitochondrial HSP75, refers to the expression product of HSPA9 gene which is a member of the HSP70 family of proteins. Mortalin is known to inhibit complement activity.

According to a specific embodiment mortalin is human mortalin such as provided in the following UniProt Accession NO: P38646 (SEQ ID NO: 23) and RefSeq Accession No: NP_004125 (SEQ ID NO: 24) or No. NM_004134 (SEQ ID NO: 25).

As used herein the phrase “having a mortalin amino acid sequence” refers to comprising at least 5 or at least 10 consecutive amino acids of the mortalin protein set forth by SEQ ID NO:24.

According to some embodiments of the invention, the peptide having at least 6, e.g., at least 7, e.g., at least 8, e.g., at least 9, e.g., at least 10, e.g., at least 11, e.g., at least 12, e.g., at least 13, e.g., at least 14, e.g., at least 15, e.g., at least 16, e.g., at least 17, e.g., at least 18, e.g., at least 19, e.g., at least 20, e.g., at least 21, e.g., at least 22, e.g., at least 23, e.g., at least 24, e.g., at least 25, e.g., at least 26, e.g., at least 27, e.g., at least 28, e.g., at least 29, e.g., at least 30, e.g., at least 31, e.g., at least 32, e.g., at least 32, e.g., at least 33, e.g., at least 34, e.g., at least 35, e.g., at least 36, e.g., at least 37, e.g., at least 38, e.g., at least 39, e.g., at least 40, e.g., at least 41, e.g., at least 42, e.g., at least 43, e.g., at least 44, e.g., at least 45, e.g., at least 46, e.g., at least 47, e.g., at least 48, e.g., at least 49, e.g., at least 50, e.g., at least 51, e.g., at least 52, e.g., at least 53, e.g., at least 54, e.g., at least 55, e.g., at least 56, e.g., at least 57, e.g., at least 58, e.g., at least 59, e.g., at least 60, e.g., at least 61, e.g., at least 62, e.g., at least 63, e.g., at least 64, e.g., at least 65, e.g., at least 66, e.g., at least 67, e.g., at least 68, e.g., at least 69, e.g., at least 70, e.g., at least 71, e.g., at least 72, e.g., at least 73, e.g., at least 74, e.g., at least 75, e.g., at least 76, e.g., at least 77, e.g., at least 78, e.g., at least 79, e.g., at least 80, e.g., at least 81, e.g., at least 82, e.g., at least 83, e.g., at least 84, e.g., at least 85, e.g., at least 86, e.g., at least 87, e.g., at least 88, e.g., at least 89, e.g., at least 90, e.g., at least 91, e.g., at least 92, e.g., at least 93, e.g., at least 94, e.g., at least 95, e.g., at least 96, e.g., at least 97, e.g., at least 98, e.g., at least 99, e.g., 100 consecutive amino acids of the mortalin protein set forth by SEQ ID NO:24.

It should be noted that the isolated peptide of some embodiments of the invention having a mortalin amino acid sequence as described above can further comprise additional amino acids which are not necessarily derived from the mortalin amino acid sequence and which can render the isolated peptide better stability, longer shelf life, improved ability to penetrate a cell and/or improved activity within the cell.

According to some embodiments of the invention, the peptide having a mortalin amino acid sequence comprises no more than 95 amino acids, e.g., no more than 90 amino acids, e.g., no more than 85 amino acids, e.g., no more than 80 amino acids, e.g., no more than 75 amino acids, e.g., no more than 70 amino acids, e.g., no more than 65 amino acids, e.g., no more than 60 amino acids, e.g., no more than 55 amino acids, e.g., no more than 50 amino acids, e.g., no more than 49 amino acids, e.g., no more than 48 amino acids, e.g., no more than 47 amino acids, e.g., no more than 46 amino acids, e.g., no more than 45 amino acids, e.g., no more than 44 amino acids, e.g., no more than 43 amino acids, e.g., no more than 42 amino acids, e.g., no more than 41 amino acids, e.g., no more than 40 amino acids, e.g., no more than 39 amino acids, e.g., no more than 38 amino acids, e.g., no more than 37 amino acids, e.g., no more than 36 amino acids, e.g., no more than 35 amino acids, e.g., no more than 34 amino acids, e.g., no more than 33 amino acids, e.g., no more than 32 amino acids, e.g., no more than 31 amino acids, e.g., no more than 30 amino acids, e.g., no more than 29 amino acids, e.g., no more than 28 amino acids, e.g., no more than 27 amino acids, e.g., no more than 26 amino acids, e.g., no more than 25 amino acids, e.g., no more than 24 amino acids, e.g., no more than 23 amino acids, e.g., no more than 22 amino acids, e.g., no more than 21 amino acids, e.g., no more than 20 amino acids, e.g., no more than 19 amino acids, e.g., no more than 18 amino acids, e.g., no more than 17 amino acids, e.g., no more than 16 amino acids, e.g., no more than 15 amino acids, e.g., no more than 14 amino acids, e.g., no more than 13 amino acids, e.g., no more than 12 amino acids, e.g., no more than 11 amino acids, e.g., no more than 100 amino acids, e.g., no more than 9 amino acids, e.g., no more than 8 amino acids.

According to some embodiments of the invention, the peptide is not a naturally occurring mortalin peptide.

According to a specific embodiment, the isolated peptide comprises a mortalin amino acid sequence with the proviso that the peptide does not comprise the amino acid sequence depicted in SEQ ID NO: 27 (KAMQDAEVSKSDIGEVI) or SEQ ID NO: 28 (QDLFGRAPSKAVNPDEA).

Mortalin comprises three major functional domains: an N-terminal Nucleotide Binding Domain (NBD) which serves as an ATPase, a C-terminal Substrate Binding Domain (SBD) and an oligomerization domain [see Kaul and Wadhwa, Mortalin Biology: Life, Stress and Death (2012) XIV, 342 p., Chapter 2, incorporated herein by reference in its entirety].

As used herein, the term “NBD” corresponds to amino acid coordinates 56-433 of RefSeq Accession No: NP_004125 (SEQ ID NO: 24) and is exemplified by SEQ ID NO: 19.

According to some embodiments of the invention, the mortalin amino acid sequence comprises at least a portion of a nucleotide binding domain (NBD) of mortalin with the proviso that the peptide does not comprise the amino acid sequence depicted in SEQ ID NO: 27 (KAMQDAEVSKSDIGEVI) or SEQ ID NO: 28 (QDLFGRAPSKAVNPDEA).

According to some embodiments of the invention, the peptide comprises the amino acid sequence set forth by SEQ ID NO:2.

According to some embodiments of the invention, the peptide consists of the amino acid sequence set forth by SEQ ID NO:2.

According to some embodiments of the invention, the peptide comprises an amino acid sequence selected from the group consisting of SEQ ID NOs: 1-13 and 29.

According to some embodiments of the invention, the peptide comprises an amino acid sequence selected from the group consisting of SEQ ID NOs: 1-13 and 29, with the proviso that the peptide does not comprise the amino acid sequence depicted in SEQ ID NO: 27 (KAMQDAEVSKSDIGEVI) or SEQ ID NO: 28 (QDLFGRAPSKAVNPDEA).

According to some embodiments of the invention, the peptide consists of the amino acid sequence selected from the group consisting of SEQ ID NOs: 1-13 and 29.

As used herein, the term “SBD” corresponds to amino acid coordinates 434-588 of RefSeq Accession No: NP_004125 (SEQ ID NO: 24) and is exemplified by SEQ ID NO: 20.

According to some embodiments of the invention, the mortalin amino acid sequence comprises at least a portion of a substrate binding domain (SBD) of mortalin.

According to some embodiments of the invention, the peptide comprises an amino acid sequence selected from the group consisting of SEQ ID NO: 14-15.

According to some embodiments of the invention, the peptide consists of the amino acid sequence selected from the group consisting of SEQ ID NO: 14-15.

As used herein, the term “oligomerization domain” corresponds to amino acid coordinates 589-679 of RefSeq Accession No: NP_004125 (SEQ ID NO: 24) and is exemplified by SEQ ID NO: 21.

According to some embodiments of the invention, the mortalin amino acid sequence comprises at least a portion of an oligomerization domain of mortalin.

According to some embodiments of the invention, the peptide comprises an amino acid sequence selected from the group consisting of SEQ ID NO: 15-18.

According to some embodiments of the invention, the peptide consists of the amino acid sequence selected from the group consisting of SEQ ID NO: 15-18.

According to some embodiments of the invention, the peptide comprises an amino acid sequence selected from the group consisting of SEQ ID NOs: 1-18 and 29.

According to some embodiments of the invention, the peptide comprises an amino acid sequence selected from the group consisting of SEQ ID NOs: 1-18 and 29, with the proviso that the peptide does not comprise the amino acid sequence depicted in SEQ ID NO: 27 (KAMQDAEVSKSDIGEVI) or SEQ ID NO: 28 (QDLFGRAPSKAVNPDEA).

According to some embodiments of the invention, the peptide consists of the amino acid sequence selected from the group consisting of SEQ ID NOs: 1-18 and 29.

As used herein, the term “complement” refers to a family of proteins which execute antibody-mediated cell death. The family includes more than 30 proteins and protein fragments including activated complement C1, C3 and C4 which are capable of activating (triggering) many cell types through specific receptors, e.g. C3a and C5a receptors, attaching (bridging) between leukocytes or lymphocytes, bacteria, immune complexes and nucleated cells and including the complement membrane attack complex (MAC)/C5b-9, which is capable of promoting cytolysis of a nucleated cell.

As described above, the isolated peptide of some embodiments of the invention is capable of killing cancer cells.

It should be noted that the killing effect on cancer cells can be dependent on complement activity or can be independent of the complement activity.

According to some embodiments of the invention, the killing of said cancer cells is complement-independent.

According to some embodiments of the invention, the peptide is capable of enhancing complement-independent cytotoxicity.

As used herein, the term “enhances complement-independent cytotoxicity” refers to an increase in complement-independent cytotoxicity in comparison to a suitable control e.g., without the peptide or with a negative control peptide. According to a specific embodiment, the increase is in at least 5%, 10%, 30%, 40% or even higher say, 50%, 60%, 70%, 80%, 90% or more than 100%. Methods of evaluating complement-independent cytotoxicity are well known in the art and include evaluating apoptotic or necrotic cell death in the absence of complement.

Methods of evaluating cell death are well known in the art and include, but not limited to Propidium Iodide (PI) inclusion, MTT test (Sigma, Aldrich St Louis, Mo., USA); the TUNEL assay [Roche, Mannheim, Germany]; the Annexin V assay [ApoAlert® Annexin V Apoptosis Kit (Clontech Laboratories, Inc., CA, USA)].

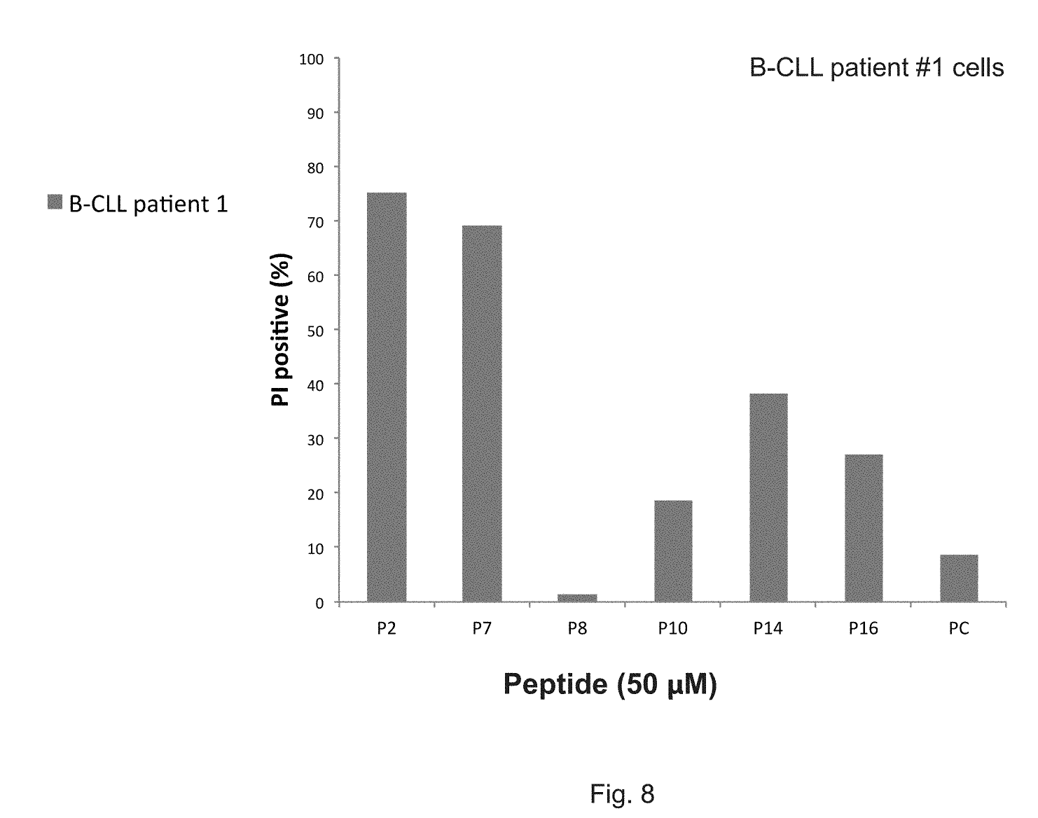

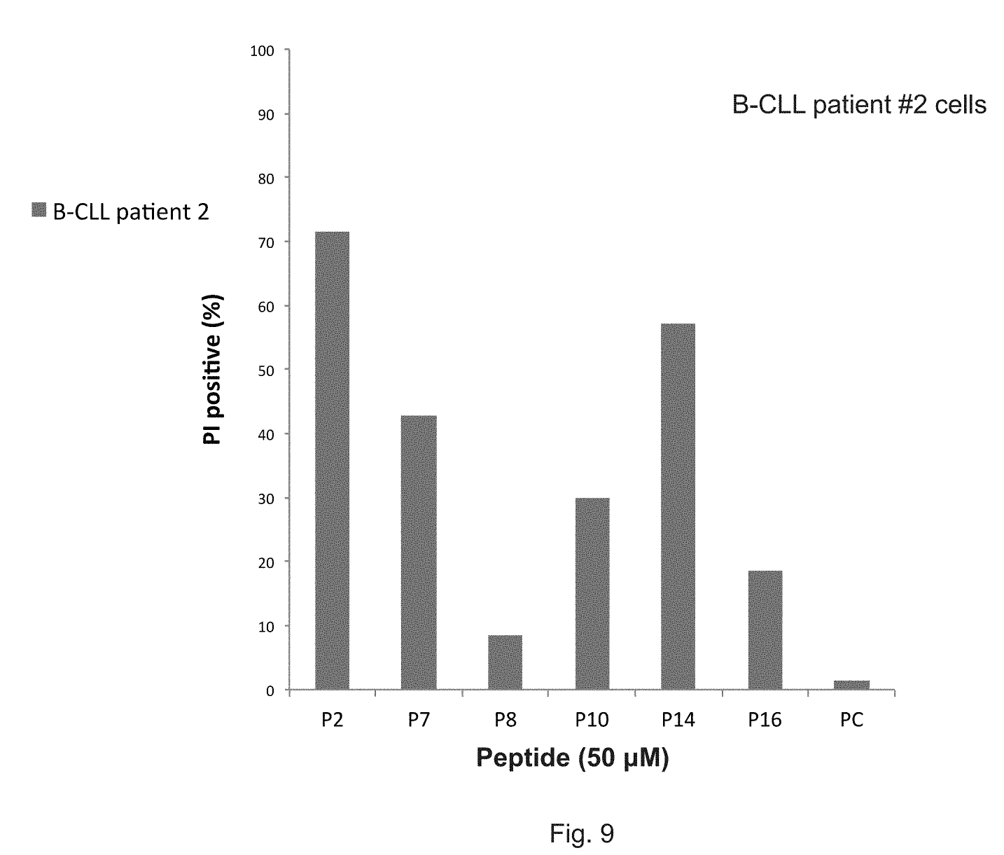

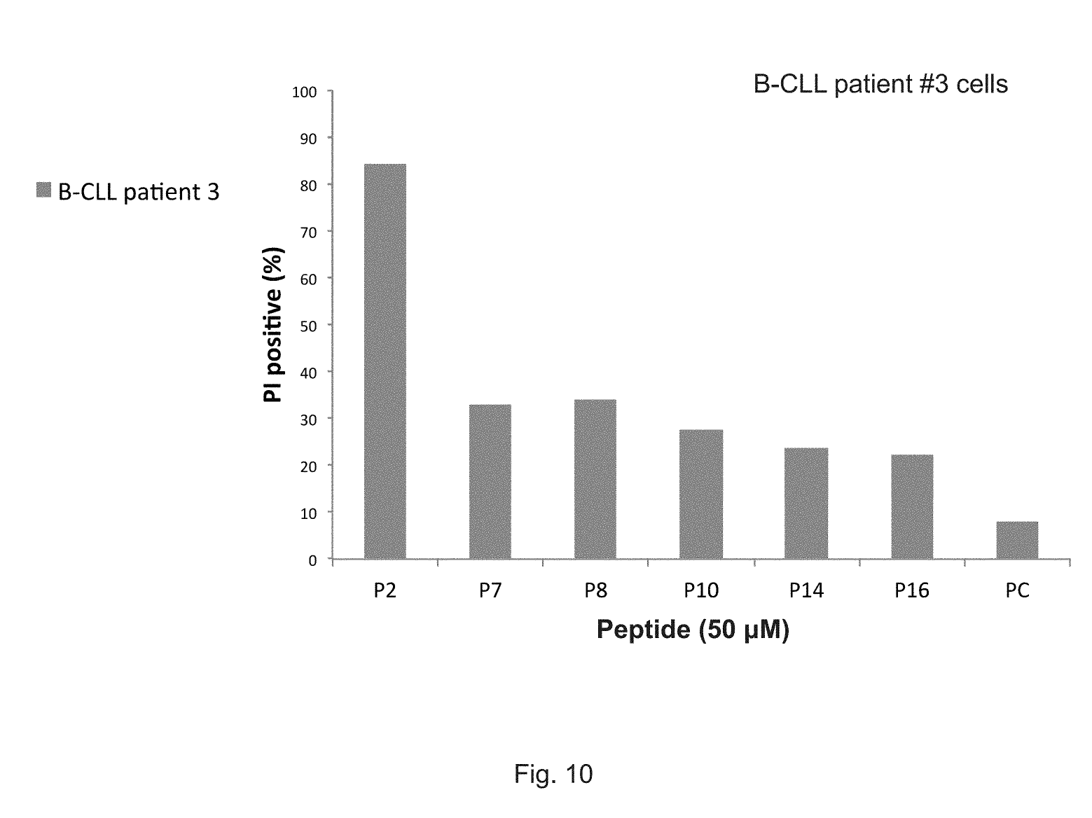

For example, as shown in FIGS. 5-7 and 8-11, the peptides of some embodiments of the invention were capable of killing cancer cells without the addition of antibody and complement, i.e., in a direct cell killing (complement independent activity). Methods of determining cell death are known in the art, and are further provided hereinbelow (Example 5 of the Examples section which follows). For example, a direct cell killing can be determined using an Annexin V/PI (Propidium Iodide) staining kit followed by flow cytometry (e.g., FACS) analysis. For example, as described in Example 5 and shown in FIGS. 5-11, peptides numbers 2, 7, 8, 10, 14 and 16 showed a direct cell killing of leukemia/lymphoma cell lines (e.g., peptide numbers 2, 7, 10, 14 and 16; FIGS. 5, 6, and 7) or of primary leukemia cells from cancer patients (e.g., peptide numbers 2, 7, 10, 14 and 16; FIG. 8, and peptide numbers 2, 7, 8, 10, 14 and 16; FIGS. 9 and 10).

Without being bound by theory it is suggested that since mortalin is a major mitochondrial chaperone, direct inhibition of mortalin can lead to mitochondrial damage (e.g., changes in mitochondrial membrane potential). Changes in mitochondrial electrochemical membrane potential gradient (MMP) can be tested using a JC-1 dye kit (Sigma-Aldrich, Rehovot, Israel) according to manufacturer's instructions. The membrane-permeant JC-1 dye is widely used in apoptosis studies to monitor mitochondrial health. JC-1 dye can be used as an indicator of mitochondrial membrane potential in a variety of cell types, including myocytes and neurons, as well as in intact tissues and isolated mitochondria. The JC-1 dye exhibits a potential-dependent accumulation in mitochondria, indicated by a fluorescence emission shift from green (˜529 nm) to red (˜590 nm). Consequently, mitochondrial depolarization is indicated by a decrease in the red/green fluorescence intensity ratio. Following is a non-limiting description of an assay which can be used to test the mitochondrial damage in cells following treatment with the isolated peptide of some embodiments of the invention. Cells (e.g., cancer cells) are incubated with the isolated peptide (e.g., at a concentration of 10-100 μM, e.g., about 50 μM) for about one hour at 37° C., following which, the cells are washed and further incubated with a JC-1 staining solution (Sigma-Aldrich, Rehovot, Israel), e.g., for about 30 minutes at 37° C., and then washed again and subjected to FACS analysis. The results measured as red mean fluorescent intensity (MFI) can be compared to cells incubated with a control peptide (e.g., a scrambled peptide, such as the peptide set forth by SEQ ID NO:30) or to cells which are not treated with any peptide. For example, peptide numbers 2 (SEQ ID NO:2) and 7 (SEQ ID NO:7) were shown capable of disrupting mitochondrial membrane potential and causing mitochondrial damage (FIG. 12 and Example 6 of the Examples section which follows). Without being bound by theory, these results may explain the direct killing effect (independent of complement activity) of the isolated peptides of some embodiments of the invention on cells such as cancer cells, and accordingly, such an assay can be used to qualify candidate peptides for their ability to cause mitochondrial toxicity and eventually to kill cells.

According to some embodiments of the invention, the ability of the isolated peptide to kill the cell is determined by presence of mitochondrial damage.

According to some embodiments of the invention, the mitochondrial damage comprises depolarization of the mitochondrial membrane.

According to some embodiments of the invention, detecting depolarization of the mitochondrial membrane is performed by monitoring the decrease in the red/green fluorescence intensity ratio emitted by the JC-1 dye.

According to some embodiments of the invention, the peptide comprises an amino acid sequence selected from the group consisting of SEQ ID NOs: 2, 7, 8, 10, 14, and 16.

According to some embodiments of the invention, the killing of the cancer cells is complement-dependent.

For example, the peptide can kill cancer cells via the activity of the complement, e.g., by enhancing the antibody-complement pathway of cell cytotoxicity.

According to some embodiments of the invention, the amino acid sequence of the isolated peptide is capable of enhancing complement activity.

According to some embodiments of the invention, the peptide is capable of enhancing complement activity.

The terms “enhances complement activity” or “enhancing complement activity”, which are interchangeably used herein, refer to an increase of at least 5% in complement activity in comparison to the complement activity in a suitable control e.g. in the absence of the peptide or in the presence of a negative control peptide. According to a specific embodiment, the increase in the complement activity is in at least 10%, 30%, 40% or even higher say, 50%, 60%, 70%, 80%, 90% or more than 100% as compared to the complement activity in the absence of the peptide or as compared to the complement activity in the presence of a negative control peptide.

Non-limiting examples of a negative control peptide include, but are not limited to a scrambled peptide of any of the isolated peptides of some embodiments of the invention. For example, a negative control peptide can be the peptide set forth by SEQ ID NO:30 (KERYNEAKEDMVA).

Enhanced complement activity may be manifested in the form of e.g. enhanced assembly of the C5b-9 complex, enhanced oligomerization of C9, enhanced stability of the C5b-9 complex (e.g., by reducing C5b-9 elimination from the plasma membrane), enhanced production of a transmembrane protein channel and/or enhanced cell death.

According to some embodiments of the invention, enhancing complement activity is via inhibiting binding of mortalin to C9.

As described above, mortalin interacts with the C8 and C9 proteins of the complement and thus causes a direct blockage of MAC incorporation into membranes.

Methods of detecting the binding between mortalin and C9 are known in the art [Pilzer et al. Int Immunol (2005) 17:1239-1248; Pilzer et al. Int J Cancer (2010). 126:1428-1435 and Saar Ray et al. J Biol Chem. (2014) 289(21):15014-22; each of which is fully incorporated herein by reference in its entirety]. For example, the ability of the isolated peptide of some embodiments of the invention to inhibit binding of mortalin to C9 can be determined using a purified human C9 protein (e.g., SEQ ID NO:31) which is attached to the surface of an ELISA plate, and which is then incubated with a labeled mortalin protein (e.g., a Histidine (His)-tagged recombinant mortalin, e.g., SEQ ID NO:24) with or without the isolated peptide of some embodiments of the invention for about one hour at 37° C. After incubation of the C9 and mortalin, the excess of the labeled mortalin and of the peptide is removed by washing the plate, and the amount of labeled mortalin which is bound to the surface-attached C9 protein is determined using an anti-His antibody (e.g., a mouse anti-His antibody). It should be noted that the anti-His antibody can be directly labeled or can be conjugated to an identifiable moiety, or it can be further incubated with a secondary antibody such as a peroxidase conjugated goat anti-mouse antibody. In either case, the binding of mortalin to C9 can be compared to a positive control, e.g., to the binding of mortalin protein (full polypeptide such as depicted in SEQ ID NO:24) to C9 in the absence of the isolated peptide of some embodiments of the invention. A non-limiting example of such an assay is shown in FIG. 1 herein.

For example, peptide numbers 2, 9, 10, 14, 15, and 16 were capable of inhibiting the binding of mortalin to C9.

According to some embodiments of the invention, the peptide comprises an amino acid sequence selected from the group consisting of SEQ ID NO: 2, 9, 10, 14, 15, and 16.

According to some embodiments of the invention, enhancing complement activity is via reducing mortalin-induced inhibition of C9 polymerization.

As used herein the term “reducing mortalin-induced inhibition of C9 polymerization” refers to an increase in C9 polymerization in comparison to a suitable control e.g. with mortalin and without the peptide or with mortalin and a negative control peptide. According to a specific embodiment, the increase is in at least 5%, 10%, 30%, 40% or even higher say, 50%, 60%, 70%, 80%, 90% or more than 100% as compared to the level of C9 polymerization in the presence of mortalin without the peptide, or as compared to the level of C9 polymerization in the presence of mortalin and a negative control peptide (e.g., a scrambled peptide, such as depicted in SEQ ID NO: 30).

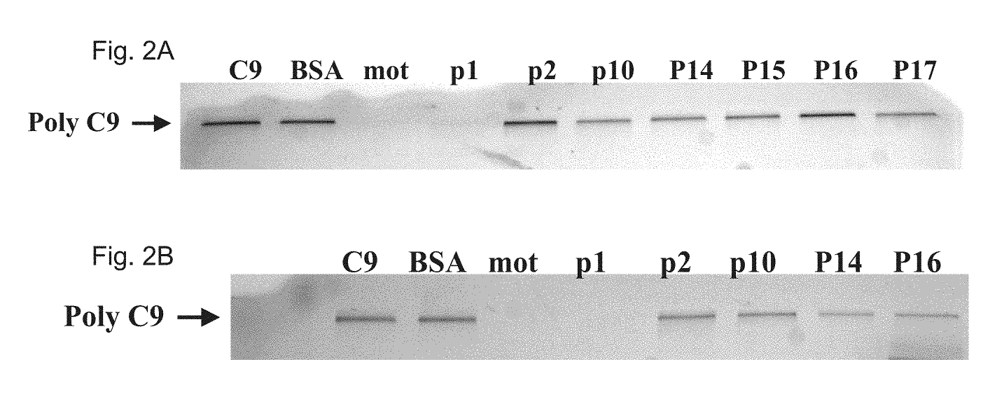

Methods of evaluating C9 polymerization are well known in the art and include evaluating purified C9 polymerization following a prolonged incubation at 37° C. or within 2 hours in the presence of metal ions such as zinc [see e.g. Tschopps, J Biol Chem. (1984) 259(16):10569-73]. A non-limiting example of such an assay is provided in FIGS. 2A-B.

Thus, the ability of the peptide of some embodiments of the invention to reduce mortalin-induced inhibition of C9 polymerization can be qualified by incubating in vitro mortalin (e.g., the recombinant polypeptide set forth by SEQ ID NO:24) and C9 (e.g., the purified polypeptide set forth by SEQ ID NO: 31) in the presence of metal ions such as zinc (e.g., 42 μM ZnCl₂ for 2 hours at 37° C.) and in the presence or absence of the isolated peptide of some embodiments of the invention, and monitoring the degree (e.g., level) of polymerized C9 (as shown in the gels of FIGS. 2A-B). The degree of C9 polymerization in the presence of C9, mortalin and the isolated peptide is then compared to the degree of C9 polymerization in the presence of C9 and mortalin.

According to some embodiments of the invention, enhancing complement activity is via reducing mortalin-induced elimination of the complement membrane attack complex C5b-9 from the plasma membrane.

As used herein the term “reducing mortalin-induced elimination of the complement membrane attack complex C5b-9 from the plasma membrane” refers to an increase in stability of the C5b-9 complex at the plasma membrane in comparison to a suitable control e.g. with mortalin and without the peptide or with mortalin and a negative control peptide. According to a specific embodiment, the increase is in at least 5%, 10%, 30%, 40% or even higher say, 50%, 60%, 70%, 80%, 90% or more than 100% as compared to the level of C5b-9 elimination in the presence of mortalin without the peptide, or as compared to the level of C5b-9 elimination in the presence of mortalin and a negative control peptide (e.g., a scrambled peptide, such as depicted in SEQ ID NO: 30).

Methods of evaluating C5b-9 elimination are well known in the art and include evaluating the quantity of C9 released from cells that have been pre-treated for 10 minutes at 37° C. with complement and thus bear C5b-9 complexes [Pilzer et al. Int Immunol (2005) 17:1239-1248 and Pilzer et al. Int J Cancer (2010). 126:1428-1435].

Thus, the ability of the peptide of some embodiments of the invention to reduce mortalin-induced C5b-9 elimination can be qualified by analyzing the quantity of C9 in the supernatant of cells (which constitutively express mortalin in the cell mainly in the mitochondria) that have been treated for 10 minutes at 37° C. with complement (e.g., normal human serum) and in the presence or absence of the isolated peptide of some embodiments of the invention, washed and then incubated for 10 minutes at 37° C. in Hank's buffer HBSS, and monitoring C9 level by Western Blotting. The degree of C9 release after treatment with complement and the isolated peptide is then compared to the degree of C9 release after treatment with complement alone.

According to some embodiments of the invention, enhancing complement activity is via enhancing complement-dependent cytotoxicity (CDC).

As used herein, the term “enhancing CDC” refers to an increase in complement dependent cytotoxicity in comparison to a suitable control e.g. without the peptide or with a negative control peptide. According to a specific embodiment, the increase is in at least 5%, 10%, 30%, 40% or even higher say, 50%, 60%, 70%, 80%, 90% or more than 100% as compared to the cytotoxicity in the absence of the peptide, or as compared to the cytotoxicity in the presence of a negative control peptide (e.g., a scrambled peptide as set forth in SEQ ID NO: 30). Methods of evaluating CDC are well known in the art and include evaluating cell death in the presence of complement and an antibody which binds the cell [see Ziporen et al. J. Immunol. (2009) 182: 515-21, Pilzer et al. Int J Cancer (2010). 126:1428-1435 and Saar Ray et al. J Biol Chem. (2014) 289(21):15014-22, each of which is fully incorporated herein by reference in its entirety].

For example, cells (e.g., cancer cells such as K562 cells) can be incubated with the peptide, an antibody (against the cancer cells, e.g., Rabbit anti-K562 polyclonal antiserum) and complement (e.g., normal human serum) and the cell death can be evaluated using any known method of detecting dead cells, such as by PI (Propidium Iodide) inclusion followed by FACS analysis, essentially as described in Example 3 and Table 4 of the Examples section which follows.

According to some embodiments of the invention, the peptide comprises an amino acid sequence selected from the group consisting of SEQ ID NO: 1, 2, 7, 9, 10, 14, 15 and 16.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)-O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2—NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom. These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| ornithine | Orn | hydroxyproline | Hyp |
| α-aminobutyric acid | Abu | aminonorbornyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl)glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3-diphenylpropyl)glycine | Nbhe | N-(N-(3,3-diphenylpropyl) | Nnbhe |
| | | carbamylmethyl-glycine | |
| 1-carboxy-1-(2,2-diphenyl | Nmbc | 1,2,3,4-tetrahydroisoquinoline-3- | Tic |
| ethylamino)cyclopropane | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The amino acids of the peptides of the present invention may be substituted either conservatively or non-conservatively.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅₋COOH]—CO— for aspartic acid. Those non-conservative substitutions which fall under the scope of the present invention are those which still constitute a peptide having neuroprotective properties.

As mentioned, the N and C termini of the peptides of the present invention may be protected by function groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

These moieties can be cleaved in vivo, either by hydrolysis or enzymatically, inside the cell. Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester.

Examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3—O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

The peptides of some embodiments of the invention are preferably utilized in a linear form, although it will be appreciated that in cases where cyclicization does not severely interfere with peptide characteristics, cyclic forms of the peptide can also be utilized.

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50.

It should be noted that in order to increase efficiency of penetration of the isolated peptide into the target cell, the isolated peptide of some embodiments of the invention can further include a cell penetration agent.

According to specific embodiments the isolated peptide is attached (either covalently or non-covalently) to a cell penetrating agent.

As used herein the phrase “penetrating agent” refers to an agent which enhances translocation of any of the attached peptide across a cell membrane.

According to specific embodiments the isolated peptide is covalently attached to the cell penetrating agent.

According to specific embodiments the cell penetrating agent is a peptide agent and is attached to the isolated peptide (either directly or non-directly) via a peptide bond.

Typically, peptide penetrating agents (CPPs) have an amino acid composition containing either a high relative abundance of positively charged amino acids such as lysine or arginine, or have sequences that contain an alternating pattern of polar/charged amino acids and non-polar, hydrophobic amino acids. Non-limiting examples of CPPs that can enter cells in a non-toxic and efficient manner and may be suitable for use with some embodiments of the invention include TAT (transcription activator from HIV-1), pAntp (also named penetratin, Drosophila antennapedia homeodomain transcription factor) and VP22 (from Herpes Simplex virus). Protocols for producing CPPs-cargos conjugates and for infecting cells with such conjugates can be found, for example L Theodore et al. [The Journal of Neuroscience, (1995) 15(11): 7158-7167], Fawell S, et al. [Proc Natl Acad Sci USA, (1994) 91:664-668], and Jing Bian et al. [Circulation Research. (2007) 100: 1626-1633].

According to some embodiments of the invention, the cell penetrating agent comprises an amino acid sequence derived from the HIV TAT polypeptide.

According to some embodiments of the invention, the amino acid sequence derived from the HIV TAT polypeptide is depicted in SEQ ID NO: 26.

The peptides of the present invention may also comprise non-amino acid moieties, such as for example, hydrophobic moieties (various linear, branched, cyclic, polycyclic or hetrocyclic hydrocarbons and hydrocarbon derivatives) attached to the peptides; non-peptide penetrating agents; various protecting groups, especially where the compound is linear, which are attached to the compound's terminals to decrease degradation. Chemical (non-amino acid) groups present in the compound may be included in order to improve various physiological properties such; decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity and the like.

Attaching the amino acid sequence component of the peptides of the invention to other non-amino acid agents may be by covalent linking, by non-covalent complexion, for example, by complexion to a hydrophobic polymer, which can be degraded or cleaved producing a compound capable of sustained release; by entrapping the amino acid part of the peptide in liposomes or micelles to produce the final peptide of the invention. The association may be by the entrapment of the amino acid sequence within the other component (liposome, micelle) or the impregnation of the amino acid sequence within a polymer to produce the final peptide of the invention.

According to some embodiments of the invention, the isolated peptide is comprised in a liposome.

Liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. The liposomes may be prepared by any of the known methods in the art [Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 September; 64(1-3):35-43]. The liposomes may be positively charged, neutral or negatively charged. For Mononuclear Phagocyte System (MPS) uptake, the liposomes can be hydrophobic since hydrophilic masking of the liposome membrane (e.g., by use of polyetheleneglycol-linked lipids and hydrophilic particles) may be less prone to MPS uptake. It is also preferable that the liposomes do not comprise sterically shielded lipids such as ganglioside-GM₁ and phosphatidylinositol since these lipids prevent MPS uptake.

The liposomes may be a single lipid layer or may be multilamellar. If the therapeutic agent is hydrophilic, its delivery may be further improved using large unilamellar vesicles because of their greater internal volume. Conversely, if the therapeutic agent is hydrophobic, its delivery may be further improved using multilamellar vesicles. Alternatively, the therapeutic agent (e.g. oligonucleotide) may not be able to penetrate the lipid bilayer and consequently would remain adsorbed to the liposome surface. In this case, increasing the surface area of the liposome may further improve delivery of the therapeutic agent. Suitable liposomes in accordance with the invention are non-toxic liposomes such as, for example, those prepared from phosphatidyl-choline phosphoglycerol, and cholesterol. The diameter of the liposomes used can range from 0.1-1.0 microns. However, other size ranges suitable for phagocytosis by phagocytic cells may also be used. For sizing liposomes, homogenization may be used, which relies on shearing energy to fragment large liposomes into smaller ones. Homogenizers which may be conveniently used include microfluidizers produced by Microfluidics of Boston, Mass. In a typical homogenization procedure, liposomes are recirculated through a standard emulsion homogenizer until selected liposomes sizes are observed. The particle size distribution can be monitored by conventional laser beam particle size discrimination. Extrusion of liposomes through a small-pore polycarbonate membrane or an asymmetric ceramic membrane is an effective method for reducing liposome sizes to a relatively well defined size distribution. Typically, the suspension is cycled through the membrane one or more times until the desired liposome size distribution is achieved. The liposomes may be extruded through successively smaller pore membranes to achieve a gradual reduction in liposome size.

Liposomes can be used for in vivo delivery of the isolated peptide of some embodiments of the invention or of a nucleic acid construct encoding same to target cells. For example, the cationic lipid formulation 3 beta [N-(N′,N′-Dimethylaminoethane)-Carbamoyl] Cholesterol (DC-Chol) is a non-viral delivery agent which can be used to target of the isolated peptide of some embodiments of the invention or of a nucleic acid construct encoding same into cells of interest (e.g., cancerous cells). For example, allogeneic and xenogeneic MHC DNA-liposome complexes were successfully employed in a phase I study of immunotherapy of cutaneous metastases of human carcinoma using the DC-Chol/DOPE cationic liposomes (see for example, Hui K M, Ang P T, Huang L, Tay S K., 1997, Gene Ther. 1997, 4(8):783-90; Serikawa T., et al., 2006, Journal of Controlled Release, 2006 Apr. 26; [Epub ahead of print]).

The liposomes can be administered directly into the tumor cells or can be administered intravenously and be directed to the cells-of-interest using a cell specific recognition moiety such as a ligand, antibody or receptor capable of specifically binding to the cell-of-interest. For example, in order to direct the liposomes to cancerous cells of an epithelial origin (e.g., breast cancer cells), the liposomes can include a ligand that can specifically recognize the cancereous cells due to overexpression of the receptor for this specific ligand. For example, one such ligand can be the keratinocyte growth factor (KGF or FGF7) molecule which is specific for cells of epithelial origin. Thus, KGF can be directed to tumors such as endometrial carcinoma or pancreatic carcinoma where the KGF receptor is overexpressed (Visco, V., et al., 1999, Expression of keratinocyte growth factor receptor compared with that of epidermal growth factor receptor and erbB-2 in endometrial adenocarcinoma, Int. J. Oncol., 15: 431-435; Siegfried, S., et al., 1997, Distinct patterns of expression of keratinocyte growth factor and its receptor in endometrial carcinoma, Cancer, 79: 1166-1171). Similarly other ligands such as EGF can be used to target lyposomes into tumors where the EGF receptor is overexpressed such as glyomas and endometrial carcinomas (for a review see: Normanno,N., et al., 2005, The ErbB receptors and their ligands in cancer: an overview, Curr. Drug Targets. 6:243-257).

According to some embodiments of the invention, the isolated peptide is recombinantly expressed from a nucleic acid construct comprising a polynucleotide which comprises a nucleic acid sequence encoding the isolated peptide of some embodiments of the invention and a promoter sequence for directing transcription of the polynucleotide sequence in the cell in a constitutive or inducible manner.

The nucleic acid construct of some embodiments of the invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

According to some embodiments of the invention, the polynucleotide of some embodiments of the invention, preferably cloned into the nucleic acid construct of some embodiments of the invention, can be used for genetically directing the production of the isolated peptide of some embodiments of the invention in a target cell.

The polynucleotide of some embodiments of the invention can be introduced into cells by any one of a variety of known methods within the art. Such methods can be found generally described in Sambrook et al., [Molecular Cloning: A Laboratory Manual, Cold Springs Harbor Laboratory, New York (1989, 1992)]; Ausubel et al., [Current Protocols in Molecular Biology, John Wiley and Sons, Baltimore, Md. (1989)]; Chang et al., [Somatic Gene Therapy, CRC Press, Ann Arbor, MI (1995)]; Vega et al., [Gene Targeting, CRC Press, Ann Arbor Mich. (1995)]; Vectors [A Survey of Molecular Cloning Vectors and Their Uses, Butterworths, Boston Mass. (1988)] and Gilboa et al. [Biotechniques 4 (6): 504-512 (1986)] and include, for example, stable or transient transfection, lipofection, electroporation and infection with recombinant viral vectors. In particular, see U.S. Pat. No. 4,866,042 for vectors involving the central nervous system and also U.S. Pat. Nos. 5,464,764 and 5,487,992 for positive-negative selection methods for inducing homologous recombination.

An advantageous approach for introducing a polynucleotide of some embodiments of the invention into cells is by using a viral vector. Viral vectors offer several advantages including higher efficiency of transformation, and targeting to, and propagation in, specific cell types. Viral vectors can also be modified with specific receptors or ligands to alter target specificity through specific cell receptors, such as neuronal cell receptors (for example, refer to Kaspar B K. et al., 2002. Mol Ther. 5:50-6).

The great majority of current clinical gene therapy trials worldwide involve viral vectors derived from retroviruses (including lentiviruses), adenoviruses, adeno-associated viruses, herpes viruses and poxviruses (Walther and Stein 2000). Retroviral vectors represent one class of vectors suitable for use with some embodiments of the invention. Defective retroviruses are routinely used in transfer of genes into mammalian cells [for review see Miller, A. D., Blood 76: 271 (1990)]. A recombinant retrovirus including a polynucleotide encoding the peptide of some embodiments of the invention can be constructed using well known molecular techniques. Portions of the retroviral genome can be removed to render the retrovirus replication defective and the replication defective retrovirus can then packaged into virions, which can be used to infect target cells through the use of a helper virus and while employing standard techniques. Protocols for producing recombinant retroviruses and for infecting cells in-vitro or in-vivo with such viruses can be found in, for example, Ausubel et al., [eds, Current Protocols in Molecular Biology, Greene Publishing Associates, (1989)]. Retroviruses have been used to introduce a variety of genes into many different cell types, including neuronal cells, epithelial cells endothelial cells, lymphocytes, myoblasts, hepatocytes and bone marrow cells.

Lentiviral vector constructs have proven to be very productive in terms of transduction due to their ability to infect both replicating and non-replicating cells. The increased use of lentiviral vector constructs in established and novel research applications makes it essential for laboratory workers to understand and protect themselves from related exposure hazards.

Another suitable expression vector may be an adenovirus vector. The adenovirus is an extensively studied and routinely used gene transfer vector. Key advantages of an adenovirus vector include relatively high transduction efficiency of dividing and quiescent cells, natural tropism to a wide range of epithelial tissues and easy production of high titers [Russel, W. C. [J. Gen. Virol. 81: 57-63 (2000)]. The adenovirus DNA is transported to the nucleus, but does not integrate thereinto. Thus the risk of mutagenesis with adenoviral vectors is minimized, while short term expression is particularly suitable for treating cancer cells. Adenoviral vectors used in experimental cancer treatments are described by Seth et al. [Adenoviral vectors for cancer gene therapy. In: P. Seth (ed.) Adenoviruses: Basic biology to Gene Therapy, Landes, Austin, Tex., (1999) pp. 103-120].

A suitable viral expression vector may also be a chimeric adenovirus/retrovirus vector which combines retroviral and adenoviral components. Such vectors may be more efficient than traditional expression vectors for transducing tumor cells [Pan et al., Cancer Letters 184: 179-188 (2002)].

A specific example of a suitable viral vector for introducing and expressing the polynucleotide sequence of some embodiments of the invention in an individual is the adenovirus-derived vector Ad-TK. This vector expresses a herpes virus thymidine kinase (TK) gene for either positive or negative selection and includes an expression cassette for desired recombinant sequences. This vector can be used to infect cells that have an adenovirus receptor which includes most cancers of epithelial origin (Sandmair et al., 2000. Hum Gene Ther. 11:2197-2205).

Features that limit expression to particular cell types can also be included. Such features include, for example, promoter and regulatory elements that are specific for the desired cell type. Secretion signals generally contain a short sequence (7-20 residues) of hydrophobic amino acids. Secretion signals are widely available and are well known in the art, refer, for example to von Heijne [J. Mol. Biol. 184:99-105 (1985)] and Lej et al., [J. Bacteriol. 169: 4379 (1987)].

The recombinant vector can be administered in several ways. If viral vectors are used the procedure can take advantage of their target specificity and consequently, such vectors do not have to be administered locally. However, local administration can provide a quicker and more effective treatment. Administration of viral vectors can also be performed by, for example, intravenous or subcutaneous injection into a subject. Following injection, the viral vectors will circulate until they recognize host cells with appropriate target specificity for infection.

According to an aspect of some embodiments of the invention, there is provided an antibody or fragments thereof comprising an antigen recognition domain having an amino acid sequence which binds a mortalin peptide and enhances complement-dependent cytotoxicity (CDC), wherein the mortalin peptide is selected from the group consisting of SEQ ID NOs: 1-10, 13-18 and 29.

According to some embodiments of the invention, the mortalin peptide is set forth by SEQ ID NO:2.

According to some embodiments of the invention, the antibody is a monoclonal antibody.

As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

As mortalin is localized intracellularly, an antibody or antibody fragment capable of specifically binding mortalin is typically an intracellular antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

According to specific embodiments the antibody comprises an antibody constant region which enables initiation of the classical pathway of complement activation.

As used herein, the term “antibody constant region” refers to the non-variable part of the antibody molecule that is capable of modulating immune cell activity. The antibody constant region enabling initiation of the classical pathway of complement activation is preferably the constant region of Ig(mu) or Ig(gamma), the heavy chains of antibodies having the IgM or IgG isotype, respectively. It will be appreciated that an antibody having a constant region capable of initiating the classical pathway of complement activation will facilitate membrane attack complex assembly at the cell surface of the targeted cells (e.g. pathological cells; e.g. cancer cells) and will result in concomitant complement-mediated cytolysis of the cells.

The isolated peptides and antibodies of the present invention may be used in inhibiting mortalin activity, killing a cell and in treating a disease associated with a pathological cell in a subject.

According to an aspect of some embodiments of the invention there is provided a method of inhibiting mortalin activity, the method comprising contacting cells which express mortalin with the isolated peptide of some embodiments of the invention or with the antibody of some embodiments of the invention thereby inhibiting mortalin activity.

According to some embodiments of the invention, inhibition of mortalin activity can promote complement independent cell death via apoptosis. Assays for testing complement activity and cell death are well known in the art [see Ziporen et al. J. Immunol. (2009) 182: 515-21, Pilzer et al. Int J Cancer (2010). 126:1428-1435 and Saar Ray et al. J Biol Chem. (2014) 289(21):15014-22, each of which is fully incorporated herein by reference in its entirety].

According to some embodiments of the invention, the mortalin activity is complement dependent.

According to some embodiments of the invention, the mortalin activity is complement independent.

According to an aspect of some embodiments of the invention there is provided a method of killing a cell, the method comprising contacting a cell which expresses mortalin with the isolated peptide of some embodiments of the invention or the antibody of some embodiments of the invention, thereby killing the cell.

According to some embodiments of the invention, the killing is complement dependent.

According to some embodiments of the invention, the killing is complement independent.

According to some embodiments of the invention, the contacting is effected in-vivo.

According to some embodiments of the invention, the contacting is effected in-vitro or ex-vivo.

According to some embodiments of the invention, the cell is a pathological cell.

As used herein, the term “pathological” when relating to a pathological cell population of the present invention refers to a cell population whose elimination in a subject of the present invention having a disease associated with such a cell population can be used to treat the disease in the subject. The pathological cell population may be any nucleated cell population derived from an organism which expresses a mortalin.

According to an aspect of some embodiments of the invention there is provided a method of treating a disease associated with a pathological cell population in a subject in need thereof, the method comprising administering to the subject a therapeutically effective amount of the isolated peptide of some embodiments of the invention or the antibody of some embodiments of the invention, thereby treating the disease associated with a pathological cell population.

The term “treating” refers to inhibiting or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology. According to specific embodiments, the subject is an organism having an immune system capable of CDC.

According to specific embodiments the subject is being treated with therapeutics or the pathology, such as immunotherapy, chemotherapy and/or radiotherapy, as further described hereinbelow.

According to some embodiments of the invention, the method further comprising administering to the subject an antibody capable of specifically binding the pathological cell population.

According to an aspect of some embodiments of the invention there is provided a use of the isolated peptide of some embodiments of the invention or the antibody of some embodiments of the invention for the manufacture of a medicament identified for the treatment of a disease associated with a pathological cell population.

According to some embodiments of the invention, the use further comprises an antibody capable of specifically binding the pathological cell population.

According to some embodiments of the invention, the disease associated with a pathological cell population is selected from the group consisting of cancer, an infectious disease, an autoimmune disease and a transplantation-related disease.

According to some embodiments of the invention, the disease associated with the pathological cell population is cancer.

According to some embodiments of the invention, the pathological cell is cancer cell.

Non-limiting examples of cancers which can be treated by the method of the present invention include any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute—megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

Precancers are well characterized and known in the art (refer, for example, to Berman J J. and Henson D E., 2003. Classifying the precancers: a metadata approach. BMC Med Inform Decis Mak. 3:8). Classes of precancers amenable to treatment via the method of the invention include, but are not limited to, acquired small or microscopic precancers, acquired large lesions with nuclear atypia, precursor lesions occurring with inherited hyperplastic syndromes that progress to cancer, and acquired diffuse hyperplasias and diffuse metaplasias. Examples of small or microscopic precancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Non-limiting examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Non-limiting examples of acquired diffuse hyperplasias and diffuse metaplasias include AIDS, atypical lymphoid hyperplasia, Paget's disease of bone, post-transplant, lymphoproliferative disease and ulcerative colitis.

According to some embodiments of the invention, the cancer is lymphoma or leukemia.

According to some embodiments of the invention, the disease associated with a pathological cell population is an infectious disease.

Specific examples of intracellular pathogens infections which may be treated according to the teachings of the present invention include, but are not limited to, infections by viral pathogens, intracellular mycobacterial pathogens (such as, for example, Mycobacterium tuberculosis), intracellular bacterial pathogens (such as, for example, Listeria monocytogenes), or intracellular protozoan pathogens (such as, for example, Leishmania and Trypanosoma).

Specific types of viral pathogens causing infectious diseases treatable according to the teachings of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to the teachings of the present invention include, but are not limited to, human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to some embodiments of the invention, the disease associated with a pathological cell population is an autoimmune disease.

Specific examples of antibody-mediated autoimmune diseases which may be treated according to the teachings of the present invention include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

According to some embodiments of the invention, the disease associated with a pathological cell population is a transplantation-related disease.

Specific examples of transplantation-related diseases which may be treated according to the teachings of the present invention include but are not limited to graft rejection, chronic graft rejection, subacute graft rejection, hyperacute graft rejection, acute graft rejection, allograft rejection, xenograft rejection and graft-versus-host disease (GVHD).

The isolated peptides and antibodies of the present invention can be used to treat diseases or conditions associated with a pathological cell alone or in combination with other established or experimental therapeutic regimen for such disorders. Thus, according to some embodiments of the invention there are provided methods of enhancing therapeutic treatment of a disease associated with a pathological cell (e.g. cancer). The methods are effected by administering to a subject in need thereof, in combination with the therapeutic treatment, the isolated peptides or the antibodies disclosed herein. It will be appreciated that such synergistic activity of combined treatment with additional therapeutic methods or compositions has the potential to significantly reduce the effective clinical doses of such treatments, thereby reducing the often devastating negative side effects and high cost of the treatment.

Therapeutic regimen suitable for combination with the isolated peptides and antibodies of some embodiments of the invention include, but are not limited to immunotherapy, chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy.

According to some embodiments the immunotherapy comprises an antibody capable of specifically binding the pathological cell population. According to some embodiments the antibody specifically binds a molecule expressed on the surface of the pathological cells or specific biological ligands of cell surface molecules of the pathological cells.

According to some embodiments of the invention, the antibody capable of specifically binding the pathological cell population comprises an antibody constant region capable of initiating a CDC.

The isolated peptides, the nucleic acid construct encoding same and/or the antibodies of some embodiments of the invention can be administered to an organism per se, or in a pharmaceutical composition where it is mixed with suitable carriers or excipients.

According to an aspect of some embodiments of the invention there is provided a pharmaceutical composition comprising as an active ingredient the isolated peptide of some embodiments of the invention or the antibody of some embodiments of the invention, and a pharmaceutically acceptable carrier or diluent.

According to some embodiments of the invention, the pharmaceutical composition further comprises an antibody capable of specifically binding a pathological cell.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the isolated peptide, the nucleic acid construct encoding same and/or the antibody accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., a disease associated with a pathological cell; e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Also provided is an article of manufacture packaging the isolated peptide or the antibody of some embodiments of the invention and an antibody capable of specifically binding a pathological cell population in single or separate containers identified for use in the treatment of a disease associated with the pathological cell population.

According to some embodiments of the invention, the peptide of some embodiments of the invention or the antibody of some embodiments of the invention and the antibody capable of specifically binding the pathological cell population are packaged in separate containers.

According to specific embodiments, the peptide of some embodiments of the invention or the antibody of some embodiments of the invention and the antibody capable of specifically binding a pathological cell population are co-formulated.

The article may be accompanied by instructions for use.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Peptides synthesis—18 mortalin peptides (SEQ ID NO: 1-18; see Table 3 below) were synthesized according to an amino acid sequence predicted by bioinformatics analysis to represent epitopes involved in mortalin interactions with its co-binders, such as C9. Each of the peptides is expected to mimic a mortalin binding site and to modify its pro-survival activities. The peptides are located at the Nucleotide Binding Domain [NBD, amino acids 56-433 in human mortalin sequence (SEQ ID NO: 19)], which serves as ATPase, the Substrate Binding Domain [SBD, amino acids 434-588 in human mortalin sequence (SEQ ID NO: 20)] or the postulated oligomerization domain [also denoted herein as “lid”, amino acids 589-679 (SEQ ID NO: 21)]. Specifically, peptides no. 1-13 are located in the NBD domain, peptide no. 14 is located in the SBD domain, peptides no: 16-18 are located in the oligomerization domain while peptide no. 15 includes amino acids flanking the SBD and the oligomerization domains. The peptides were obtained from Genemed Synthesis, San Antonio, Tex. or from Mimotopes, Australia.

TABLE 3
Amino acid sequences of the
18 selected mortalin peptides

| | | | Amino acids |
|---|---|---|---|
| | | | position |
| Peptide | SEQ ID | Amino | (Coordinates) on |
| NO: | NO: | acid sequence | human mortalin |
| 1 | 1 | KLYSPSQIGAFVLMK | 159-180 |
| | | MKETAEN | |
| 2 | 2 | PSQIGAFVLMKMKET | 163-182 |
| | | AENYL | |
| 3 | 3 | ATKDAGQISG | 204-213 |
| 4 | 4 | PAYFNDSQRQATKDA | 194-211 |
| | | GQI | |
| 5 | 5 | DSQRQATKDAGQI | 199-211 |
| 6 | 6 | EPTAAALAYGLDKS | 222-235 |
| 7 | 7 | GEDFDQALLRHIVKE | 275-294 |
| | | FKRET | |
| 8* | 8 | NMALQRVREAAEKAK | 302-319 |
| | | SEL | |
| 9 | 9 | PKHLNMKLTRAQFEG | 339-361 |
| | | IVTDLIRR | |
| 10 | 10 | RAQFEGIVTDLIRRT | 348-364 |
| | | IA | |
| 11 | 11 | PCQKAMQD | 365-372 |
| 12 | 12 | MPKVQQTVQDLFGR | 392-405 |
| 13 | 13 | PDEAVAIGAAIQGGV | 413-429 |
| | | LA | |
| 14 | 14 | MVKNAEKYAEEDR | 561-573 |
| 15 | 15 | RKKERVEAVNMAEGI | 574-603 |
| | | IHDTETKMEEFKDQL | |
| 16 | 16 | FKDQLPADECNKLKE | 599-623 |
| | | EISKMRELLA | |
| 17 | 17 | NKLKEEISKMRELLA | 609-632 |
| | | RKDSETGEN | |
| 18 | 18 | NIRQAASSLQQASLK | 632-657 |
| | | LFEMAYKKMAS | |

The amino acids positions refers to the Human mortalin polypeptide sequence provided in GenBank Accession No: NP_004125 (SEQ ID NO: 24).
*it should be noted that peptide no. 8 was synthesized with a Serine residue instead of a Cysteine residue at position 16 in order to avoid a possible di-sulfide bond, however, the native amino acid sequence at this position [i.e., the amino acid sequence depicted by NMALQRVREAAEKAKCEL (SEQ ID NO: 29; marked as peptide no. 8′) is also contemplated for use by the invention.

### Example 1

### Identification of Mortalin Peptide which Inhibit or Promote Binding of Mortalin to C9

Experimental Results

The effect of the peptides on mortalin-C9 binding—The potential of the peptides to inhibit C9-mortalin binding was evaluated in an ELISA assay in which C9 was attached overnight to wells of 96 wells plate, followed by addition of the peptide and then His-tagged recombinant mortalin. The amount of bound mortalin was quantified colorimetrically as described in description of the drawings section with respect to FIG. 1. Wells containing only C9 and mortalin without peptide served as positive control. Of the 18 mortalin peptides, two were shown to promote C9-mortalin binding (peptide no: 7 located at the NBD domain and peptide no: 17 located at the lid domain) as demonstrated in FIG. 1. In contrast, peptides no: 2, 9, 10, 14, 15 and 16 were shown to function as inhibitors of mortalin-C9 binding (FIG. 1). Their combination did not elicit any additive effect on mortalin-C9 binding inhibition (data not shown).

### Example 2

### Mortalin Peptides Affect C9 Polymerization

Experimental Results

The effect of the peptides on C9 polymerization—Formation of the complement membrane pores requires that multiple C9 molecules undergo polymerization and generate the doughnut-shape transmembrane complex; thus the effect of the peptides on C9 polymerization was tested. C9 polymerization occurs during C5b-9 generation [Podack and Tschopp, Proc Natl Acad Sci USA. (1982) 79(2):574-8]. Purified C9 polymerizes after a prolonged incubation at 37° C. or within 2 hours in the presence of metal ions such as zinc [Tschopp, J Biol Chem. (1984) 259(16):10569-73]. C9 was mixed with peptides and 42 μM ZnCl₂ for 2 hours and the samples were analyzed by SDS-PAGE, on a 2.5-14% acrylamide gradient gel. The large (>1,000 kDa) polymerized C9 band (poly C9) appears on the top part of the gel (FIGS. 2A and 2B). As shown in FIGS. 2A and 2B (representing two independent experiments), mortalin (marked as “mot” in FIGS. 2A-B) completely inhibited C9 polymerization, in agreement with the fact that mortalin regulates the C5b-9 deposition and confers resistance to CDC. Peptide no. 1 (p1) inhibited C9 polymerization to the same extent as mortalin, however, all other tested peptides (peptides no: 2, 10, 14, 15, 16 and 17) inhibited C9 polymerization but significantly less than mortalin.

### Example 3

### Identification of Mortalin Peptides which Enhance Complement-Dependent Cytotoxicity

Experimental Methods

K562 cells were either pre-incubated with peptides (60 minutes at 37° C.) and then treated with Rabbit anti-K562 polyclonal antiserum (Ab) and normal human serum (as a source for complement) or treated with Ab and complement in presence of the peptide [Ziporen et al. J. Immunol. (2009) 182: 515-21]. Cell death was measured by PI (Propidium Iodide) inclusion and FACS analysis, after 1 hour treatment at 37° C.

Experimental Results

The effect of the peptides on complement-dependent cytotoxicity—The next objective was to determine which of the peptides could alter cell death and sensitize cancer cells to complement-dependent cytotoxicity (CDC). Thus, cells were incubated with the peptides prior to antibody and complement treatment (denoted herein as pre-incubation) or treated concomitantly with antibody and complement in the presence of the peptides (denoted herein as without pre-incubation).

As demonstrated in Table 4 below, peptides no. 1, 2, 7, 9, 10, 14, 15 and 16 significantly enhanced cell death of K562 cells induced by antibody (Ab) and complement in at least one of the settings i.e. with or without pre-incubation. Most of the peptides showed a bell shape dose effect and had a reduced effect at a higher dose (5 mM).

TABLE 4
The effect of the peptides on CDC

| Peptide | | |
|---|---|---|
| No. | CDC with preincubation* | CDC without preincubation* |
| 1 | 1.6 (1 mM) | 2.6 (0.1 mM) |
| 2** | 0.4-06 (1 mM) | 4.1 (1 mM) |
| 7 | 0.6-0.8 (1 mM) | 1.5-3.1 (1-5 mM) |
| 9 | 2.2 (1 mM) | 1.2 (1 mM) |
| 10 | 2.0 (1 mM); 1.3 (5 mM) | 1.0-1.2 (0.1-1 mM); 0.7 (5 mM) |
| 14 | 3-4.1 (5 mM) | 2.3 (1 mM); 1.5 (5 mM) |
| 15 | 1.6 (5 mM) | 1.3-3.5 (1 mM); 0.8-1.4 (5 mM) |
| 16 | 1.2-3.1 (5 mM) | 4.0-4.9 (1 mM) |
| 17 | 0.8 (1 mM) | 0.8-1.4 (1 mM) |

Table 4: *K562 cells were either pre-incubated with peptides (60 minutes at 37° C.) and then treated with Ab (antibody) and complement or treated with Ab and complement in presence of the peptide [Ziporen et al. J. Immunol. (2009) 182: 515-21]. Cell death was measured by PI (Propidium Iodide) inclusion (FACS analysis), after 1 hour at 37° C. CDC is calculated relative to control cells treated in the absence of peptides (control CDC = 1.0). Enhancement factor: CDC > 1.0; Inhibition factor: CDC < 1.0.
**might have been only partially soluble when used.

It should be noted that in cases where the peptide was incubated with the cells prior to addition of the antibody and complement (serum) (noted as “preincubation”) cell cytotoxicity begins as complement independent, and then, upon addition of the antibody and complement can continue as complement dependent. On the other hand, when the cells interact with the peptide, antibody and complement without preincubation of the cells and peptide (noted as “without preincubation”), cell cytotoxicity is entirely complement dependent.

### Example 4

### Peptide Ability to Enter Cells

Experimental methods—K562 cells were incubated with biotin-tagged peptides for 30 minutes at 37° C. and then washed. Cell lysates were prepared, diluted in buffer and attached overnight at 4° C. to wells of an ELISA plate. Next, biotin-peptide in each well was quantified with Streptavidin-peroxidase by ELISA.

Experimental Results

In the next step, the capacity of the peptides to penetrate the cells was examined using biotinylated peptides. Based on the amount of peptide found inside the cells (as analysed by ELISA using Streptavidin-peroxidase), the peptides were divided into high, medium and low penetrants.
High penetrants: peptides no: 2, 7, 15 and 16.
Medium penetrants: peptides no: 1 and 17.
Low penetrants: peptides no: 9, 10 and 14.

### Example 5

### Complement Independent Cytotoxic Effect

Experimental Results

The complement independent cytotoxic effect of the peptides—To address the toxic effect of the peptides as single agents, the present inventor tested the effect of the peptides no. 14 and 16 on K562 and Raji human cancer cells. The cells were incubated with the peptides for 24-48 hours in growth medium and then tested for apoptosis using an Annexin V/PI (Propidium Iodide) staining kit followed by FACS analysis. Peptides no: 14 and 16 induced direct apoptosis in Raji and K562 cells in the absence of antibody and complement. As shown in FIG. 3A, 48 hours following culture of Raji or K562 cells with 2 mM mortalin peptides, approximately 100% of Raji cells and 40% of K562 cells died.

Since peptide no: 14 showed low cell penetration capacity, the present inventor has synthesized this peptide with and without a cell penetrating sequence [HIV TAT-derived sequence (designated “TAT” herein) depicted by YMGRKKRRQRRR (SEQ ID NO:26)], and compared the toxicity of the native (SEQ ID NO: 14) and the TAT-modified (MVKNAEKYAEEDRYMGRKKRRQRRR, SEQ ID NO: 22) peptides. As shown in FIG. 4, the TAT modification markedly enhanced the capacity of peptide no: 14 to induce apoptosis in K562 and Raji cells as a stand-alone treatment.

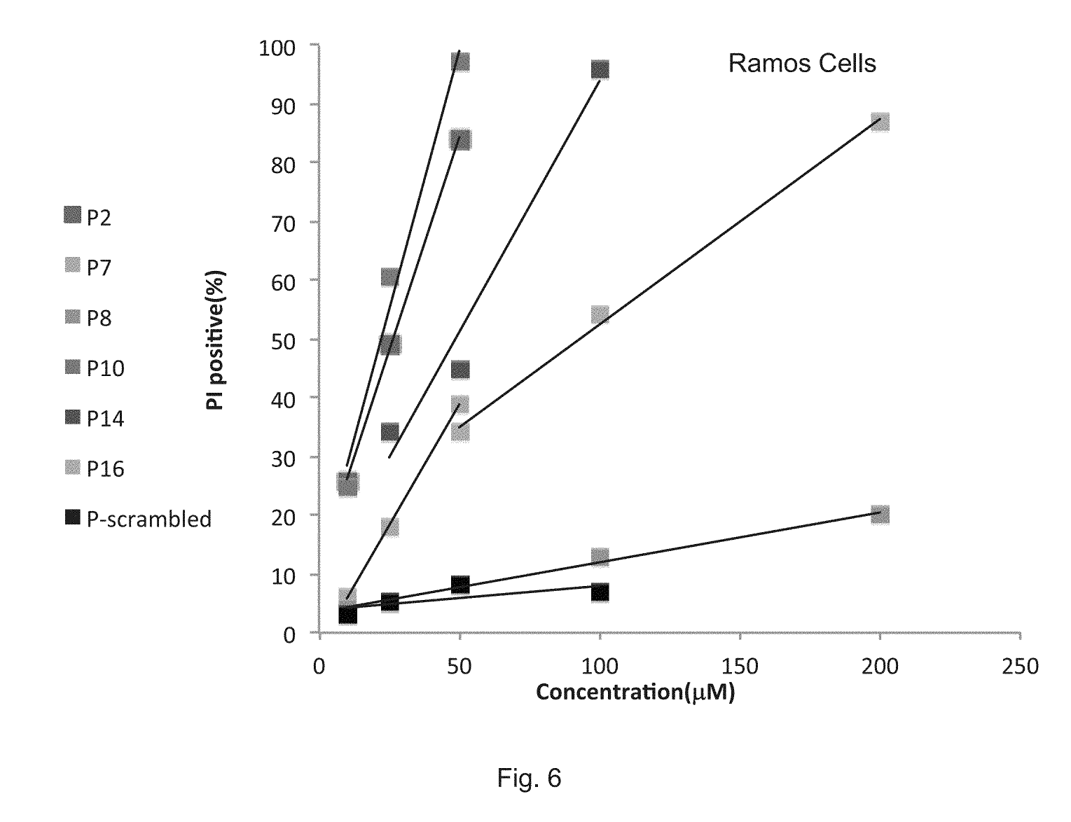

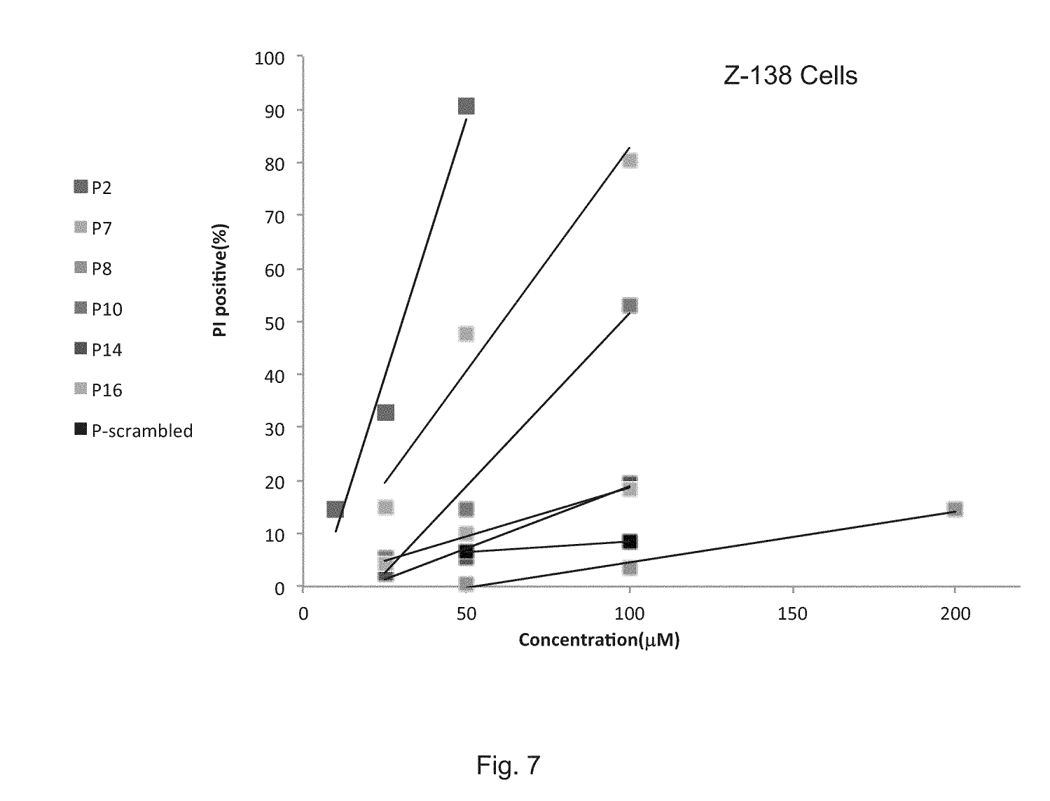

The peptides of some embodiments of the invention exhibit a direct cell killing effect on leukemia and lymphoma cell lines—To further test the ability of the mortalin peptides to kill cancer cells, the peptides no: 2, 7, 8, 10, 14 and 16 and a control peptide with a sequence scrambled were synthesized with an added TAT sequence (SEQ ID NO:26) at their C-terminal. The toxic effect of these peptides was tested on 3 leukemia/lymphoma cell lines Raji (FIG. 5), Ramos (FIG. 6) and Z-138 (FIG. 7). The cells were treated for 24 hours with the peptides and cell death was determined by propidium iodide labeling and analysis by flow cytometry. As shown in FIGS. 5-7, peptides nos. 2, 7, 10, 14 and 16 exhibit a significant cell killing effect on all leukemia and lymphoma cell lines tested.

The peptides of some embodiments of the invention exhibit a direct cell killing effect on primary leukemia cell preparations—Next, the toxic effect of peptides nos: 2, 7, 8, 10, 14 and 16 and a control peptide with TAT sequence at their C-terminal was tested on 3 primary B-CLL leukemia cell preparations. Blood drawing from B-CLL patients was approved by the Helsinki Committee of Rabin Medical Center, Petach Tikvah, Israel. Peripheral blood was collected with EDTA from three B-CLL patients and from one healthy volunteer who signed a letter of consent (named patients #1, 2, 3 and 4 and “Healthy”). The blood cells were fractionated over Ficoll gradients and mononuclear cells were purified. By cell counting it became clear that >95% of the cells separated from the B-CLL patients (28-107 million cells were purified from each ml blood) were leukemia cells. From the healthy donor, 1.4 million mononuclear cells were purified from each ml blood). The cells were treated for 20 hours with the peptides at 50 mM concentration and cell death was determined by propidium iodide labeling and analysis by flow cytometry. The results obtained with the various cells are presented in FIGS. 8-11. As shown in FIGS. 8-11, peptides nos. 2, 7, 8, 10, 14 and 16 exhibit a cell killing effect on various primary leukemia cells. It is noted that peptide no. 2 exhibit the most significant toxic effect on all three primary leukemia cells.

Determination of the LD50 concentration of the peptides of some embodiments of the invention—The concentration of the peptides (in microM) that causes 50% cell death (LD50) in Raji, Ramos and Z-138 cells was determined from FIGS. 5-7 (Table 5). As can be seen, peptide #2>peptide #7>peptide #14 expressed the highest level of toxicity to those 3 cancer cell lines.

TABLE 5
LD50 (in μM) of the mortalin peptides

| Peptide# | Raji | Ramos | Z-138 |
|---|---|---|---|
| P2 | 28 | 26 | 30 |
| P7 | 40 | 63 | 61 |
| P8 | 1728 | 547 | 574 |
| P10 | 130 | 22 | 97 |
| P14 | 87 | 48 | 232 |
| P16 | 123 | 92 | 262 |

In summary, Table 6 below illustrates the relative effects of some of the peptides on mortalin-C9 binding, C9 polymerization, complement-dependent cytotoxicity and complement independent cytotoxicity (direct toxicity).

TABLE 6
Summary of peptides in vitro effects

| | Mortalin-C9 | C9 | Effect on | Direct |
|---|---|---|---|---|
| Peptide # | binding | polymerization | CDC | toxicity |
| Mortalin | nt | ↓↓↓ | ↓↓↓ | — |
| 1 | — | ↓↓ | ↑ | nt |
| 2 | ↓ | — | ↑↓ | ↑↑↑ |
| 7 | ↑ | nt | ↑↓ | ↑↑ |
| 8 | — | nt | nt | ↑ (*)— |
| 9 | ↓ | nt | ↑ | nt |
| 10 | ↓ | ↓ | ↑↓ | ↑↑ |
| 14 | ↓ | ↓ | ↑↑ | ↑↑ |
| 15 | ↓ | ↓ | ↑ | nt |
| 16 | ↓ | ↓ | ↑↑ | ↑ |
| 17 | ↑ | ↓ | — | nt |

Table 6: * Saar Ray M. et al. J. Biol. Chem. (2014) 289(21): 15014-22; Pilzer D. et al. (2010) Int. J. Cancer 126: 1428-35. The effect is depicted by arrows, the description is as follows: “↑”, enhance; “↓“, inhibit; “↑↓“, effect depends on dose/time; “—“, no effect; “nt”, not tested.
* a cytotoxic effect was observed in primary leukemia cell preparations from 2 B-CLL patients but not in cell lines.

### Example 6

### The Peptides of Some Embodiments of the Invention Cause Damage to Mitochondria

Since mortalin is a major mitochondrial chaperone, the effect of the isolated peptides of some embodiments of the invention on the mitochondrial electrochemical membrane potential gradient (MMP) was tested using a JC-1 dye kit (Sigma-Aldrich, Rehovot, Israel). JC-1 dye exhibits a potential-dependent accumulation in mitochondria, indicated by a fluorescence emission shift from green (˜529 nm) to red (˜590 nm). Consequently, mitochondrial depolarization is indicated by a decrease in the red/green fluorescence intensity ratio.

Experimental Results

Ramos cells were incubated for 1 hour at 37° C. with either 50 μM of scrambled peptide (PC; SEQ ID NO: 30) or with 50 μM peptide number 2 (P2, SEQ ID NO: 2) or 7 (P7, SEQ ID NO:7), or were left untreated. Following incubation, the cells were washed and a staining solution was added for 20 minutes at 37° C. Cells were washed and suspended in JC-1 staining buffer (Sigma) and analyzed by FACS. Red mean fluorescence intensity (MFI) was quantified. As shown in FIG. 12, upon treating the cells with P2 or P7 for 1 hour at 37° C., mitochondrial membrane potential dissipated (red MFI decreased) relative to that of cells treated with a scrambled peptide (PC). These results show that the isolated peptide of some embodiments of the invention can disrupt MMP, which leads to mitochondrial damage (mitochondrial toxicity) and predispose the cells to death. Without being bound to theory, these results may explain the direct killing effect (independent of complement activity) of the isolated peptides of some embodiments of the invention on cells such as cancer cells. In addition, such an assay can be used to qualify candidate peptides for their ability to cause mitochondrial toxicity and eventually to kill cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An isolated peptide comprising no more than 100 amino acids having a mortalin amino acid sequence and being capable of killing cancer cells.

2. An isolated peptide comprising no more than 100 amino acids having a mortalin amino acid sequence and being capable of enhancing complement activity.

3. The isolated peptide of claim 1, with the proviso that the peptide does not comprise the amino acid sequence depicted in SEQ ID NO: 27 (KAMQDAEVSKSDIGEVI) or SEQ ID NO: 28 (QDLFGRAPS KAVNPDEA).

4. The isolated peptide of claim 1, wherein said killing of said cancer cells is complement-dependent.

5. The isolated peptide of claim 4, wherein said amino acid sequence is capable of enhancing complement activity.

6. The isolated peptide of claim 1, wherein said killing of said cancer cells is complement-independent.

7. The isolated peptide of claim 2, wherein said enhancing complement activity is via inhibiting binding of mortalin to C9.

8. The isolated peptide of claim 2, wherein said enhancing complement activity is via reducing mortalin-induced inhibition of C9 polymerization.

9. The isolated peptide of claim 2, wherein said enhancing complement activity is via enhancing complement-dependent cytotoxicity (CDC).

10. The isolated peptide of claim 1, wherein said peptide is capable of enhancing complement-independent cytotoxicity.

11. The isolated peptide of claim 1, wherein said mortalin amino acid sequence comprises at least a portion of a nucleotide binding domain (NBD) of mortalin with the proviso that the peptide does not comprise the amino acid sequence depicted in SEQ ID NO: 27 (KAMQDAEVSKSDIGEVI) or SEQ ID NO: 28 (QDLFGRAPSKAVNPDEA).

12. (canceled)

13. The isolated peptide of claim 11, wherein said peptide comprises an amino acid sequence selected from the group consisting of SEQ ID NOs: 1-13 and 29.

14. The isolated peptide of claim 1, wherein said mortalin amino acid sequence comprises at least a portion of a substrate binding domain (SBD) of mortalin.

15. (canceled)

16. The isolated peptide of claim 1, wherein said mortalin amino acid sequence comprises at least a portion of an oligomerization domain of mortalin.

17. (canceled)

18. The isolated peptide of claim 1, wherein said peptide comprises an amino acid sequence selected from the group consisting of SEQ ID NOs: 1-18 and 29.

19. The isolated peptide of claim 1, wherein said peptide comprises an amino acid sequence selected from the group consisting of SEQ ID NOs: 2, 7, 8, 10, 14 and 16.

20-21. (canceled)

22. The isolated peptide of claim 1, wherein said peptide comprises no more than 30 amino acids.

23. The isolated peptide of claim 1, wherein said peptide is attached to a cell penetrating agent.

24. An antibody comprising an antigen recognition domain having an amino acid sequence which binds a mortalin peptide and enhances complement-dependent cytotoxicity (CDC), wherein said mortalin peptide is selected from the group consisting of SEQ ID NOs: 1-10, 13-18 and 29.

25. The antibody of claim 24, wherein said mortalin peptide is set forth by SEQ ID NO:2.

26. A method of inhibiting mortalin activity, the method comprising contacting cells which express mortalin with the isolated peptide of claim 1, thereby inhibiting mortalin activity.

27. A method of killing a cell, the method comprising contacting a cell which expresses mortalin with the isolated peptide of claim 1, thereby killing the cell.

28. The method of claim 26, wherein said mortalin activity is complement dependent.

29. The method of claim 26, wherein said mortalin activity is complement independent.

30-31. (canceled)

32. A method of treating a disease associated with a pathological cell population in a subject in need thereof, the method comprising administering to the subject a therapeutically effective amount of the isolated peptide of claim 1, thereby treating the disease associated with a pathological cell population.

33. The method of claim 32, further comprising administering to the subject an antibody capable of specifically binding said pathological cell population.

34-35. (canceled)

36. A pharmaceutical composition comprising as an active ingredient the isolated peptide of claim 1, and a pharmaceutically acceptable carrier or diluent.

37. The pharmaceutical composition of claim 36, further comprises an antibody capable of specifically binding a pathological cell.

38. An article of manufacture identified for treatment of a disease associated with a pathological cell population comprising packaging material packaging the isolated peptide of claim 1 and an antibody capable of specifically binding the pathological cell population.

39. The method of claim 32, wherein said disease associated with a pathological cell population is selected from the group consisting of cancer, an infectious disease, an autoimmune disease and a transplantation-related disease.

40. (canceled)
